SEQUENCE LISTING (11 sequences)

SEQ ID NO: 1 - protein, 225 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 2 - protein, 5 aa, synthetic construct
  REGION 1..5: note = synthetic construct
YSWIN

SEQ ID NO: 3 - protein, 17 aa, synthetic construct
  REGION 1..17: note = synthetic construct
RIFPGDGDTDYNGKFKG

SEQ ID NO: 4 - protein, 10 aa, synthetic construct
  REGION 1..10: note = synthetic construct
NVFDGYWLVY

SEQ ID NO: 5 - protein, 16 aa, synthetic construct
  REGION 1..16: note = synthetic construct
RSSKSLLHSNGITYLY

SEQ ID NO: 6 - protein, 7 aa, synthetic construct
  REGION 1..7: note = synthetic construct
QMSNLVS

SEQ ID NO: 7 - protein, 9 aa, synthetic construct
  REGION 1..9: note = synthetic construct
AQNLELPYT

SEQ ID NO: 8 - protein, 119 aa, synthetic construct
  REGION 1..119: note = synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 9 - protein, 115 aa, synthetic construct
  REGION 1..115: note = synthetic construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTV

SEQ ID NO: 10 - protein, 297 aa, Homo sapiens
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 11 - protein, 254 aa, Homo sapiens
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

(19) United States
(12) Patent Application Publication
(10) Pub. No.: US 2024/0058443 A1
(43) Pub. Date: Feb. 22, 2024

(54) PREVENTION OR MITIGATION OF NK CELL ENGAGING AGENT-RELATED ADVERSE EFFECTS

(71) Applicant: Hoffmann-La Roche Inc., Little Falls, NJ (US)

(72) Inventors: Hélène Cécile HAEGEL, Illkirch-Graffenstaden (FR); Christian KLEIN, Bonstetten (CH); Gabrielle LECLERCQ, Zürich (CH); Alberto TOSO, Basel (CH); Tina ZIMMERMANN, Unterengstringen (CH)

(73) Assignee: Hoffmann-La Roche Inc., Little Falls, NJ (US)

(21) Appl. No.: 18/491,669

(22) Filed: Oct. 20, 2023

Related U.S. Application Data
(63) Continuation of application No. PCT/EP2022/060469, filed on Apr. 21, 2022.

(30) Foreign Application Priority Data
Apr. 23, 2021 (EP) 21170037.2

(51) Int. Cl. A61K 39/395; C07K 16/28; A61K 31/519; A61K 31/436; A61K 31/506

(52) U.S. Cl. CPC A61K 39/3955; C07K 16/283; C07K 16/2887; A61K 31/519; A61K 31/436; A61K 31/506; C07K 2317/24

(57) ABSTRACT

The present invention relates to the prevention or mitigation of adverse effects related to NK cell engaging agents, such as cytokine-related infusion reactions. Specifically, the invention relates to the prevention or mitigation of such side effects using an inhibitor of Src, JAK and/or mTOR.

### CROSS REFERENCE TO RELATED APPLICATIONS

This application is a continuation of International Application No. PCT/EP2022/060469, filed Apr. 21, 2022, claiming priority to European Application No. 21170037.2 filed Apr. 23, 2021, each of which is incorporated herein by reference in its entirety.

### SEQUENCE LISTING

This application contains a Sequence Listing which has been submitted electronically in XML format and is hereby incorporated by reference in its entirety. Said XML copy, created on Sep. 26, 2023, is named P36849-US Sequence Listing.xml and is 14.1 KB bytes in size.

### Field of the Invention

The present invention relates to the prevention or mitigation of adverse effects related to natural killer (NK) cell engaging agents, such as cytokine-related infusion reactions. Specifically, the invention relates to the prevention or mitigation of such side effects using an inhibitor of Src, JAK and/or mTOR.

### Background

Natural killer (NK) cell engaging agents such as effector-enhanced antibodies hold great promise as cancer immunotherapeutics. However, treatment with NK cell engaging agents, such as effector-enhanced antibodies, may be associated with safety liabilities due to cytokine release. A common adverse effect reported for NK cell engaging agents, for example the antibody obinutuzumab, are infusion-related reactions (IRRs) which may be caused by cytokine release. IRR symptoms are diverse, including fever, chills, headache, nausea, hypotension, dispnoea, fatigue, and/or diarrhea, and may be life-threatening (see e.g. Snowden et al., International Journal of Nursing Practice (2015) 21 (Suppl. 2), 15-27). Approaches to mitigate these serious toxicities are greatly needed.

The Src kinase inhibitor dasatinib was identified as a potent candidate for prevention or mitigation of adverse effects, in particular Cytokine Release Syndrome (CRS), caused by T cell engaging agents such CAR-T cells (Weber et al., Blood Advances (2019) 3, 711-7; Mestermann et al., Sci Transl Med (2019) 11, eaau5907) as well as T cell bispecific antibodies (TCBs) (Leclercq et al., J Immunother Cancer (2020) 8 (Suppl 3): A690 (abstract 653)). Dasatinib switches off CAR-T cell functionality as well as TCB-induced T cell functionality entirely, without differentiation between desired and undesired activity of these agents.

A way to prevent or mitigate adverse effects of NK cell engaging agents while preserving their therapeutic efficacy would be highly desirable.

### Description of the Invention

The present inventors have found that inhibitors of Src kinase (Src), Janus kinase (JAK) and/or mammalian target of rapamycin (mTOR) signaling may be used to mitigate CRS by NK cell engaging therapies. Src inhibitors such as dasatinib, mTOR inhibitors such as temsirolimus, sirolimus and everolimus, and JAK inhibitors such as ruxolitinib, were found to potently prevent cytokine release induced by an NK cell engaging antibody, while retaining target cell killing mediated by such antibody. The results provide evidence that the mechanisms of cytokine release related to the onset of IRRs and target cell killing mediated by NK cell engaging agents can be uncoupled, and suggest the use of Src, mTOR and/or JAK inhibitors as attractive strategy for the mitigation of IRRs associated with NK cell engaging therapies.

Accordingly, in a first aspect, the present invention provides a natural killer (NK) cell engaging agent for use in the treatment of a disease in an individual, wherein said treatment comprises
(a) the administration of the NK cell engaging agent to the individual, and
(b) the administration of an inhibitor of Src kinase (Src), Janus kinase (JAK) and/or mammalian target of rapamycin (mTOR) signaling to the individual.

The invention further provides the use of an NK cell engaging agent in the manufacture of a medicament for the treatment of a disease in an individual, wherein said treatment comprises
(a) the administration of the NK cell engaging agent to the individual, and
(b) the administration of an inhibitor of Src, JAK and/or mTOR signaling to the individual.

The invention also provides a method for treatment of a disease in an individual, wherein said method comprises
(a) the administration of an NK cell engaging agent to the individual, and
(b) the administration of an inhibitor of Src, JAK and/or mTOR signaling to the individual.

According to any of the above aspects, the administration of the inhibitor of Src, JAK and/or mTOR signaling may be for the prevention or mitigation of an adverse effect related to the administration of the NK cell engaging agent.

In another aspect, the invention provides an inhibitor of Src, JAK and/or mTOR signaling for use in the prevention or mitigation of an adverse effect related to the administration of an NK cell engaging agent to an individual.

The invention further provides the use of an inhibitor of Src, JAK and/or mTOR signaling in the manufacture of a medicament for the prevention or mitigation of an adverse effect related to the administration of an NK cell engaging agent.

The invention also provides a method for preventing or mitigating an adverse effect related to the administration of an NK cell engaging agent to an individual, comprising the administration of an inhibitor of Src, JAK and/or mTOR signaling to the individual.

The NK cell engaging agent for use, inhibitor of Src, JAK and/or mTOR signaling for use, uses or methods described above and herein, may incorporate, singly or in combination, any of the features described in the following (unless the context dictates otherwise).

Terms are used herein as generally used in the art, unless otherwise defined herein.

In some aspects, the inhibitor of Src, JAK and/or mTOR signaling is a Src inhibitor. In more specific aspects, the inhibitor of Src, JAK and/or mTOR signaling is a Src kinase inhibitor, particularly a small molecule Src kinase inhibitor. In particular aspect, the inhibitor of Src, JAK and/or mTOR signaling is dasatinib.

Dasatinib is a Src kinase inhibitor, sold under the brand name Sprycel® (among others), for the treatment of certain cases of chronic myelogenous leukemia (CML) and acute lymphoblastic leukemia (ALL). Its CAS number, IUPAC name and chemical structure are shown below.

CAS number: 302962-49-8

IUPAC name: N-(2-chloro-6-methylphenyl)-2[[6[4-(2-hydroxyethyl)-1-piperazinyl]-2-methyl-4-pyrimidinyl]amino]-5-thiazole carboxamide monohydrate

### Chemical Structure:

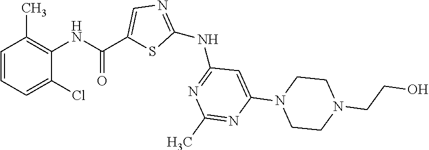 [SMILES: Cc1nc(Nc2ncc(C(=O)Nc3c(C)cccc3Cl)s2)cc(N2CCN(CCO)CC2)n1]

In some aspects, the inhibitor of Src, JAK and/or mTOR signaling is an mTOR inhibitor. In more specific aspects, the inhibitor of Src, JAK and/or mTOR signaling is an mTOR kinase inhibitor, particularly a small molecule mTOR kinase inhibitor.

“mTOR” stands for mammalian target of rapamycin (also known as FK506-binding protein 12-rapamycin complex-associated protein 1 (FRAP1)), and is a serine/threonine-specific protein kinase that belongs to the family of phosphatidylinositol-3 kinase (PI3K) related kinases. It serves as core component of two distinct protein complexes, mTOR complex 1 (TORC1) and mTOR complex 2 (TORC2), which regulate different cellular processes. Human mTOR is described in UniProt entry P42345 (version 218). mTOR inhibitors are compounds that inhibit mTOR. The most established inhibitors of mTOR are the so-called rapalogs, which are derivatives of rapamycin. Rapalogs include sirolimus, temsirolimus, everolimus and ridaforolimus. A second generation of mTOR inhibitors are ATP-competitive mTOR kinase inhibitors, designed to compete with ATP in the catalytic site of mTOR.

Exemplary mTOR inhibitors that might be useful in the present invention are provided in Table 1 below.

TABLE 1
mTOR inhibitors.

| INN | Structure |
|---|---|
| Sirolimus | 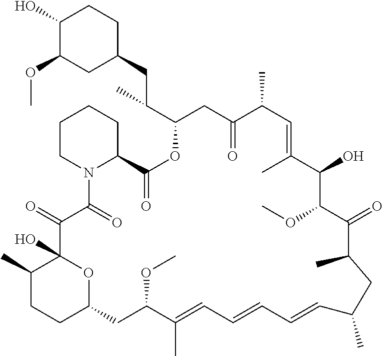 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C/1C] |
| Everolimus | 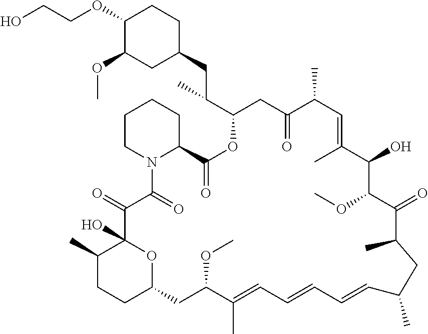 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCO)[C@H](OC)C2)CC(=O)[C@H](C)/C=C(\C)[C@@H](O)[C@@H](OC)C(=O)[C@H](C)C[C@H](C)/C=C/C=C/C=C/1C] |
| Temsirolimus | 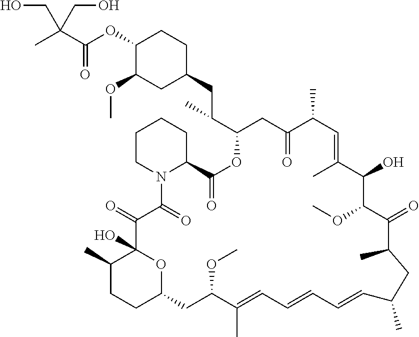 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)C(C)(CO)CO)[C@H](OC)C2)CC(=O)[C@H](C)/C=C(\C)[C@@H](O)[C@@H](OC)C(=O)[C@H](C)C[C@H](C)/C=C/C=C/C=C/1C] |
| Ridaforolimus | 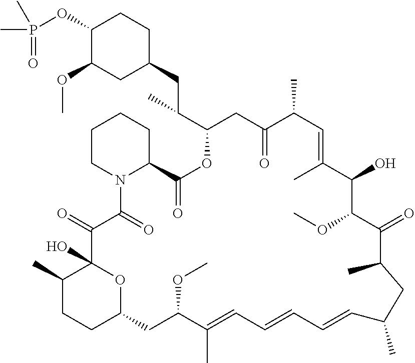 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OP(C)(C)=O)[C@H](OC)C2)CC(=O)[C@H](C)/C=C(\C)[C@@H](O)[C@@H](OC)C(=O)[C@H](C)C[C@H](C)/C=C/C=C/C=C/1C] |

In some aspects, the mTOR inhibitor is a derivative of rapamycin (also known as a rapalog).

In some aspects, the mTOR inhibitor is selected from the group consisting of sirolimus, temsirolimus, everolimus and ridaforolimus, particularly the group consisting of sirolimus, temsirolimus and everolimus.

In specific aspects, the mTOR inhibitor is sirolimus. In further specific aspects, the mTOR inhibitor is temsirolimus. In yet further specific aspects, the mTOR inhibitor is everolimus.

In some aspects, the inhibitor of Src, JAK and/or mTOR signaling is a JAK inhibitor. In more specific aspects, the inhibitor of Src, JAK and/or mTOR signaling is a JAK kinase inhibitor, particularly a small molecule JAK kinase inhibitor.

“JAK” stands for Janus kinase and refers to a family of intracellular, non-receptor tyrosine kinases that transduce cytokine-mediated signals via the JAK/STAT pathway. JAKs possess two near-identical phosphate-transferring domains, one exhibiting the kinase activity, and the other one negatively regulating the kinase activity of the first. The four JAK family members are JAK1, JAK2, JAK3 and TYK2 (tyrosine kinase 2). In particular aspects herein, JAK is JAK1 and/or JAK2 (JAK1/2). Human JAK1 and JAK2 are described in UniProt entries P23458 (version 221) and P60674 (version 224), respectively. JAK inhibitors (also sometimes referred to as jakinibs) are compounds that inhibit the activity of one or more of the JAK family of enzymes (JAK1, JAK2, JAK3, TYK2), thereby interfering with the the JAK/STAT signaling pathway.

Exemplary JAK inhibitors that might be useful in the present invention are provided in Table 2 below.

TABLE 2
JAK inhibitors.

| | Main | |
|---|---|---|
| INN | specificity | Structure |
| Ruxolitinib | JAK1, JAK2 | 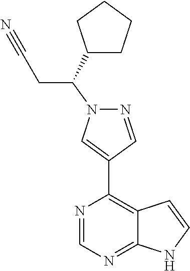 [SMILES: N#CC[C@H](C1CCCC1)n1cc(-c2ncnc3[nH]ccc23)cn1] |
| Baricitinib | JAK1, JAK2 | 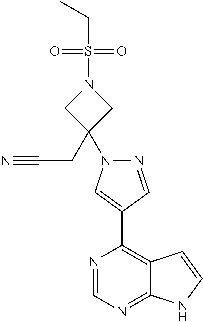 [SMILES: CCS(=O)(=O)N1CC(CC#N)(n2cc(-c3ncnc4[nH]ccc34)cn2)C1] |
| Momelotinib | JAK1, JAK2 | 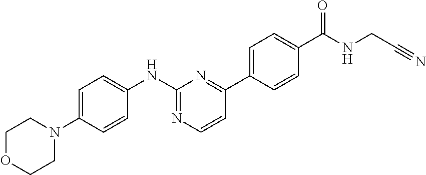 [SMILES: N#CCNC(=O)c1ccc(-c2ccnc(Nc3ccc(N4CCOCC4)cc3)n2)cc1] |
| Upadacitinib | JAK1 | 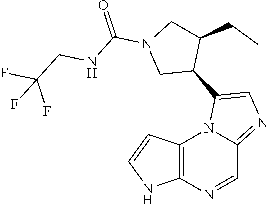 [SMILES: CC[C@@H]1CN(C(=O)NCC(F)(F)F)C[C@@H]1c1cnc2cnc3[nH]ccc3n12] |
| Filgotinib | JAK1 | 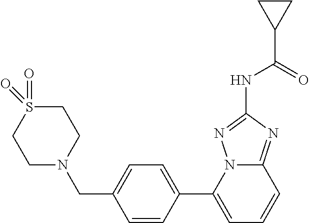 [SMILES: O=C(Nc1nc2cccc(-c3ccc(CN4CCS(=O)(=O)CC4)cc3)n2n1)C1CC1] |
| Abrocitinib | JAK1 | 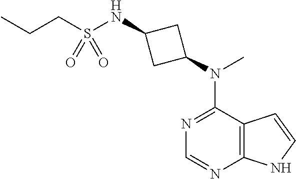 [SMILES: CCCS(=O)(=O)N[C@H]1C[C@@H](N(C)c2ncnc3[nH]ccc23)C1] |
| Itacitinib | JAK1 | 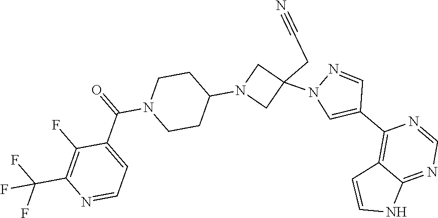 [SMILES: N#CCC1(n2cc(-c3ncnc4[nH]ccc34)cn2)CN(C2CCN(C(=O)c3ccnc(C(F)(F)F)c3F)CC2)C1] |
| Solcitinib | JAK1 | 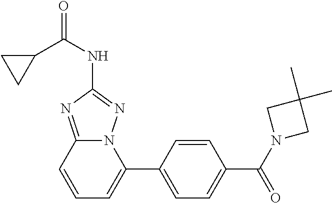 [SMILES: CC1(C)CN(C(=O)c2ccc(-c3cccc4nc(NC(=O)C5CC5)nn34)cc2)C1] |
| Oclacitinib | JAK1 | 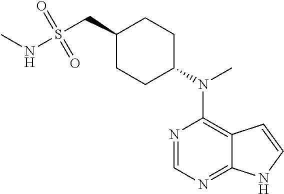 [SMILES: CNS(=O)(=O)C[C@H]1CC[C@H](N(C)c2ncnc3[nH]ccc23)CC1] |
| Fedratinib | JAK2 | 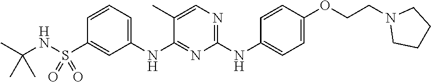 [SMILES: Cc1cnc(Nc2ccc(OCCN3CCCC3)cc2)nc1Nc1cccc(S(=O)(=O)NC(C)(C)C)c1] |
| Gandotinib | JAK2 | 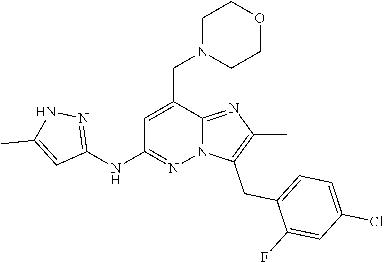 [SMILES: Cc1cc(Nc2cc(CN3CCOCC3)c3nc(C)c(Cc4ccc(Cl)cc4F)n3n2)n[nH]1] |
| Lestaurtinib | JAK2 | 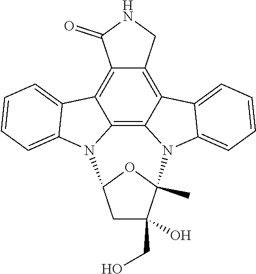 [SMILES: C[C@]12O[C@H](C[C@]1(O)CO)n1c3ccccc3c3c4c(c5c6ccccc6n2c5c31)CNC4=O] |
| Pacritinib | JAK2 | 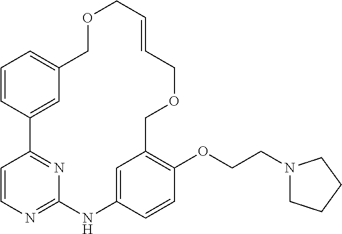 [SMILES: C1=C/COCc2cc(ccc2OCCN2CCCC2)Nc2nccc(n2)-c2cccc(c2)COC/1] |
| Peficitinib | JAK3 (pan-JAK) | 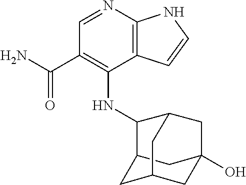 [SMILES: NC(=O)c1cnc2[nH]ccc2c1NC1C2CC3CC1CC(O)(C3)C2] |
| Tofacitinib | JAK3 (pan-JAK) | 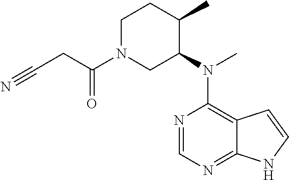 [SMILES: C[C@@H]1CCN(C(=O)CC#N)C[C@@H]1N(C)c1ncnc2[nH]ccc12] |
| Decernotinib | JAK3 | 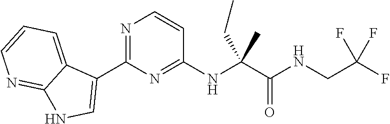 [SMILES: CC[C@@](C)(Nc1ccnc(-c2c[nH]c3ncccc23)n1)C(=O)NCC(F)(F)F] |

In some aspects, the JAK inhibitor is a JAK1 and/or JAK2 (JAK1/2) inhibitor. In some aspects, the JAK inhibitor is selected from the group consisting of ruxolitinib, baricitinib, momelotinib, upadacitinib, filgotinib, abrocitinib, itacitinib, solcitinib, oclacitinib, fedratinib, gandotinib, lestaurtinib and pacritinib.

In particular aspects, the JAK inhibitor is a JAK1 and JAK2 inhibitor. In specific such aspects, the JAK inhibitor is selected from the group consisting of ruxolitinib, baricitinib and momelotinib.

In some aspects, the JAK inhibitor is a JAK1 inhibitor. In specific such aspects, the JAK inhibitor is selected from the group consisting of upadacitinib, filgotinib, abrocitinib, itacitinib, solcitinib and oclacitinib.

In some aspects, the JAK inhibitor is a JAK2 inhibitor. In specific such aspects, the JAK inhibitor is selected from the group consisting of fedratinib, gandotinib, lestaurtinib and pacritinib.

In particular aspects, the JAK inhibitor is ruxolitinib.

In particular aspects, the inhibitor of Src, JAK and/or mTOR signaling is selected from the group consisting of dasatinib, sirolimus, temsirolimus, everolimus and ruxolitinib. In further particular aspects, the inhibitor of Src, JAK and/or mTOR signaling is selected from the group consisting of dasatinib, sirolimus and ruxolitinib.

In some aspects, (administration of) the inhibitor of Src, JAK and/or mTOR signaling causes inhibition of an activity of the NK cell engaging agent. In some aspects, (administration of) the inhibitor of Src, JAK and/or mTOR signaling does not cause inhibition of another activity of the NK cell engaging agent. In some aspects, (administration of) the inhibitor of Src, JAK and/or mTOR signaling causes inhibition of a first activity of the NK cell engaging agent but does not cause inhibition of a second activity of the NK cell engaging agent. In some of these aspects, said inhibition is a complete inhibition.

In some aspects, (administration of) the inhibitor of Src, JAK and/or mTOR signaling causes inhibition of a first activity of the NK cell engaging agent and inhibition of a second activity of the NK cell engaging agent, wherein said inhibition of the first activity is stronger than said inhibition of the second activity. In some aspects, (administration of) the inhibitor of Src, JAK and/or mTOR signaling causes inhibition of a first activity of the NK cell engaging agent and inhibition of a second activity of the NK cell engaging agent, wherein said inhibition of the first activity is a complete inhibition and said inhibition of the second activity is a partial inhibition.

“Activity” of a NK cell engaging agent refers to responses in an individual's body caused by the NK cell engaging agent. Such activity may include cellular response(s) of NK cells, particularly CD16⁺ NK cells, such as proliferation, differentiation, cytokine secretion, cytotoxic effector molecule release, cytotoxic activity, and expression of activation markers, and/or effects on target cells, particularly target cells (e.g. tumor cells) expressing the target cell antigen of the NK cell engaging agent, such as lysis of target cells.

In some aspects, (administration of) the inhibitor of Src, JAK and/or mTOR signaling causes inhibition of cytokine secretion by immune cells, particularly NK cells (induced by the NK cell engaging agent). In some aspects, said immune cells are CD16⁺ immune cells. In some aspects, said cytokine is one or more cytokine selected from the group consisting of IL-6, IFN-γ, IL-8, TNF-α, IL-2, IL-12, IL-1β, MCP-1 and IL-10, particularly the group consisting of IL-6, IFN-γ, IL-8 and TNF-α. Immune cells may include various immune cell types, such as NK cells, macrophages, monocytes, T cells etc. In some aspects, said T cells are γδ T cells. In some aspects, said inhibition is a complete inhibition.

In some aspects, (administration of) the inhibitor of Src, JAK and/or mTOR signaling does not cause inhibition of the activation of NK cells (induced by the NK cell engaging agent). In some aspects, said inhibition is a complete inhibition. In some aspects, (administration of) the inhibitor of Src, JAK and/or mTOR signaling causes inhibition of the activation of NK cells (induced by the NK cell engaging agent), wherein said inhibition is a partial inhibition.

“Activation of NK cells” or “NK cell activation” as used herein refers to one or more cellular response of an NK cell, particularly a CD16⁺ NK cell, selected from: proliferation, differentiation, cytotoxic effector molecule release, cytotoxic activity, and expression of activation markers. Suitable assays to measure NK cell activation are known in the art and described herein. In particular aspects, NK cell activation is the expression of activation markers, particularly expression of CD25 and/or CD69 (optionally as measured by flow cytometry). In particular aspects, NK cell activation is determined by measuring expression of CD25 and/or CD69 on the NK cell, e.g. by flow cytometry.

In some aspects, (administration of) the inhibitor of Src, JAK and/or mTOR signaling does not cause inhibition of the cytotoxic activity of NK cells (induced by the NK cell engaging agent). In some aspects, said inhibition is a complete inhibition. In some aspects, (administration of) the inhibitor of Src, JAK and/or mTOR signaling causes inhibition of the cytotoxic activity of NK cells (induced by the NK cell engaging agent), wherein said inhibition is a partial inhibition.

“Cytotoxic activity” of an NK cell refers to the induction of lysis (i.e. killing) of target cells by a NK cell, particularly a CD16⁺ NK cell. Cytotoxic activity typically involves degranulation of the NK cell, associated with the release of cytotoxic effector molecules such as granzyme B and/or perform from the NK cell.

In some aspects, (administration of) the inhibitor of Src, JAK and/or mTOR signaling causes inhibition of cytokine secretion by NK cells (induced by the NK cell engaging agent) but does not cause inhibition of the activation and/or the cytotoxic activity of NK cells (induced by the NK cell engaging agent). In some of these aspects, said inhibition is a complete inhibition.

In some aspects, (administration of) the inhibitor of Src, JAK and/or mTOR signaling causes inhibition of cytokine secretion by NK cells (induced by the NK cell engaging agent) and inhibition of the activation and/or the cytotoxic activity of NK cells (induced by the NK cell engaging agent), wherein said inhibition of cytokine secretion is stronger than said inhibition of activation and/or cytotoxic activity. In some aspects, (administration of) the inhibitor of Src, JAK and/or mTOR signaling causes inhibition of cytokine secretion by NK cells (induced by the NK cell engaging agent) and inhibition of the activation and/or the cytotoxic activity of NK cells (induced by the NK cell engaging agent), wherein said inhibition of cytokine secretion is a complete inhibition and said inhibition of activation and/or cytotoxic activity is a partial inhibition.

An inhibition herein may be a partial inhibition or a complete inhibition. A complete inhibition is a stronger inhibition than a partial inhibition. A partial inhibition in some aspects is an inhibition by no more than 30%, no more than 40%, no more than 50%, no more than 60%, or no more than 70%. In some aspects, a partial inhibition is an inhibition by no more than 30%. In some aspects, a partial inhibition is an inhibition by no more than 40%. In some aspects, a partial inhibition is an inhibition by no more than 50%. In some aspects, a partial inhibition is an inhibition by no more than 60%. In some aspects, a partial inhibition is an inhibition by no more than 70%. A complete inhibition in some aspects is an inhibition by at least 80%, at least 90%, or 100%. In some aspects, a complete inhibition is an inhibition by at least 80%. In some aspects, a complete inhibition is an inhibition by at least 90%. In some aspects, a complete inhibition is an inhibition by 100%. In some aspects, a partial inhibition is an inhibition by no more than 50%, and a complete inhibition is an inhibition by at least 80%. In some aspects, a complete inhibition is clinically meaningful and/or statistically significant, and/or a partial inhibition is not clinically meaningful and/or statistically significant.

In some aspects, (administration of) the inhibitor of Src, JAK and/or mTOR signaling causes reduction of the serum level of one of more cytokine in the individual. In some aspects, (administration of) the inhibitor of Src, JAK and/or mTOR signaling causes reduction of the secretion of one of more cytokine by immune cells, particularly NK cells, in the individual. In some aspects, said immune cells are CD16⁺ immune cells. In some aspects, said one or more cytokine is selected from the group consisting of IL-6, IFN-γ, IL-8, TNF-α, IL-2, IL-12, IL-1β, MCP-1 and IL-10, particularly the group consisting of IL-6, IFN-γ, IL-8 and TNF-α. Immune cells may include various immune cell types, such as NK cells, macrophages, monocytes, T cells etc. In some aspects, said T cells are γδ T cells.

In some aspects, said reduction is sustained after the inhibitor of Src, JAK and/or mTOR signaling has not been administered (to the individual) for a given amount of time. In some aspects, said amount of time is about 1 hour, 2 hours, 3 hours, 4 hours, 5 hours, 6 hours, 7 hours, 8 hours, 12 hours, 16 hours, 20 hours, 24 hours, 36 hours, 48 hours, 72 hours, or 96 hours. In some aspects, said reduction is sustained after a subsequent administration of the NK cell engaging agent. Particularly, said reduction is sustained even after administration of the inhibitor of Src, JAK and/or mTOR signaling is stopped/no further administration of the inhibitor of Src, JAK and/or mTOR signaling is made. Said reduction of the serum level/cytokine secretion is in particular as compared to the serum level/cytokine secretion in an individual (including the same individual) without administration of the inhibitor of Src, JAK and/or mTOR signaling (i.e. in such case the serum level/cytokine secretion is reduced as compared to the serum level/cytokine secretion without/before administration of the inhibitor of Src, JAK and/or mTOR signaling). Said reduction of the serum level/cytokine secretion is in particular as compared to the serum level/cytokine secretion in an individual (including the same individual) with administration (in particular first administration) of the NK cell engaging agent but without administration of the inhibitor of Src, JAK and/or mTOR signaling (i.e. in such case the serum level/cytokine secretion is reduced as compared to the serum level/cytokine secretion with/after administration of the NK cell engaging agent but without/before administration of the inhibitor of Src, JAK and/or mTOR signaling). Without said reduction, the serum level/cytokine secretion particularly may be elevated/increased in relation to the (administration of) the NK cell engaging agent. In some aspects, said reduction is clinically meaningful and/or statistically significant. In some aspects, said reduction is at least 30%, at least 40%, at least 50%, at least 60%, or at least 70%. In some aspects, said reduction is at least 30%. In some aspects, said reduction is at least 40%. In some aspects, said reduction is at least 50%. In some aspects, said reduction is at least 60%. In some aspects, said reduction is at least 70%.

In some aspects, (administration of) the inhibitor of Src, JAK and/or mTOR signaling causes inhibition of an adverse effect related to the administration of the NK cell engaging agent. In some aspects, (administration of) the inhibitor of Src, JAK and/or mTOR signaling does not cause inhibition of a desired effect related to the administration of the NK cell engaging agent. In some aspects, (administration of) the inhibitor of Src, JAK and/or mTOR signaling causes inhibition of an adverse effect related to the administration of the NK cell engaging agent but does not cause inhibition of a desired effect related to the administration of the NK cell engaging agent. In some of these aspects, said inhibition is a complete inhibition. In some of these aspects, said inhibition is clinically meaningful and/or statistically significant.

In some aspects, (administration of) the inhibitor of Src, JAK and/or mTOR signaling causes inhibition of an adverse effect related to the administration of the NK cell engaging agent and inhibition of a desired effect related to the administration of the NK cell engaging agent, wherein said inhibition of the adverse effect is stronger than said inhibition of the desired effect. In some aspects, (administration of) the inhibitor of Src, JAK and/or mTOR signaling causes inhibition of an adverse effect related to the administration of the NK cell engaging agent and inhibition of a desired effect related to the administration of the NK cell engaging agent, wherein said inhibition of the adverse effect is a complete inhibition and said inhibition of the beneficial effect is a partial inhibition. In some aspects, (administration of) the inhibitor of Src, JAK and/or mTOR signaling causes inhibition of an adverse effect related to the administration of the NK cell engaging agent and inhibition of a desired effect related to the administration of the NK cell engaging agent, wherein said inhibition of the adverse effect is a clinically meaningful and/or statistically significant inhibition and said inhibition of the beneficial effect is not a clinically meaningful and/or statistically significant inhibition.

A “desired effect” is a beneficial and desired effect resulting from medication in the treatment of an individual, herein particularly with a NK cell engaging agent, i.e. a therapeutic or prophylactic effect, such as e.g. killing of tumor cells, reduction or retardation of tumor growth, reduction of tumor volume, reduction or prevention of tumor metastasis, increase of progression-free or overall survival, alleviation of disease symptoms, and the like.

An “adverse effect”, which is sometimes also denoted as “side effect” or “adverse event” (especially in clinical studies) is a harmful and undesired effect resulting from medication in the treatment of an individual, herein particularly with a NK cell engaging agent.

According to the invention the adverse effect is related to the administration of the NK cell engaging agent. In some aspects, the adverse effect is related to the first administration of the NK cell engaging agent. In some aspects, the adverse effect occurs upon the first administration of the NK cell engaging agent. In some aspects, the adverse effect occurs predominantly or only upon the first administration of the NK cell engaging agent. In some aspects, the adverse effect occurs within 12 hours, 24 hours, 36 hours. 48 hours. 72 hours or 96 hours of the administration, particularly the first administration, of the NK cell engaging agent. In some aspects, in particular wherein only a single administration of the NK cell engaging is made (in the course of the treatment with the NK cell engaging agent), the adverse effect occurs within 3 days, 4 days, 5 days, 6 days, 7 days, 10 days, 14 days or 21 days of the administration of the NK cell engaging agent.

In some aspects, said adverse effect is an infusion-related reaction (IRR), particularly an IRR related to cytokine release.

“Infusion-related reaction” (abbreviated as “IRR”) refers to an adverse effect associated with the (intravenous) administration of a therapeutic agent (e.g. an NK cell engaging agent). IRRs always involve the immune system and are timely related to the administration of the therapeutic agent. They typically occur during or shortly after an administration of the therapeutic agent, i.e. typically within 24 hours after administration (typically intravenous infusion), predominantly at the first administration. In some instances, IRRs can also occur only later, e.g. several days after administration of the therapeutic agent. The incidence and severity typically decrease with subsequent administrations. Symptoms may range from symptomatic discomfort to fatal events, and may include fever, chills, pyrexia, hypotension, hypoxia, dyspnea, flushing, skin rash, muscle pain, tachycardia, headache, dizziness, nausea, vomiting and/or organ failure. IRRs may be graded according to severity into Grade 1 (mild) to Grade 4 (life-threatening). See e.g. Snowden et al., International Journal of Nursing Practice (2015) 21 (Suppl. 2), 15-27; Vogel, Clinical Journal of Oncology Nursing (2010) 14, E10-21). In particular aspects herein, IRRs are caused by an increase in the levels of cytokines, such tumor necrosis factor alpha (TNF-α), interferon gamma (IFN-γ), interleukin-6 (IL-6), interleukin-8 (IL-8) and others, in the blood of a subject during or shortly after (e.g. within 1 day of) administration of a therapeutic agent (e.g. an NK cell engaging agent), resulting in adverse symptoms.

In some aspects, said adverse effect is fever, hypotension and/or dispnoea.

In some aspects, said adverse effect is an elevated serum level of one of more cytokine. Said elevated serum level is in particular as compared to the serum level in a healthy individual, and/or the serum level in an individual (including the same individual) without administration of the NK cell engaging agent (i.e. in such case the serum level is elevated as compared to the serum level without administration of the NK cell engaging agent). In some aspects, said one or more cytokine is selected from the group consisting of IL-6, IFN-γ, IL-8, TNF-α, IL-2, IL-12, IL-1β, MCP-1 and IL-10, particularly the group consisting of IL-6, IFN-γ, IL-8 and TNF-α.

In some aspects, administration of the inhibitor of Src, JAK and/or mTOR signaling is upon (clinical) manifestation of the adverse effect (in the individual). Said administration may be, for example, within about 1 hour, 2 hours, 3 hours, 4 hours, 5 hours, 6 hours, 7 hours, 8 hours, 12 hours, 16 hours, 20 hours or 24 hours after manifestation of the adverse effect (i.e. the occurrence clinical symptoms of the side effect, such as fever). In some aspects, administration of the inhibitor of Src, JAK and/or mTOR signaling is in response to the (clinical) manifestation of the adverse effect (in the individual).

In some aspects, administration of the inhibitor of Src, JAK and/or mTOR signaling is before the administration of the NK cell engaging agent. In some aspects, administration of the inhibitor of Src, JAK and/or mTOR signaling is concurrent to the administration of the NK cell engaging agent. In some aspects, administration of the inhibitor of Src, JAK and/or mTOR signaling is after the administration of the NK cell engaging agent. Where administration of the inhibitor of Src, JAK and/or mTOR signaling is before or after the administration of the NK cell engaging agent, such administration of the inhibitor of Src, JAK and/or mTOR signaling may be, for example, within about 1 hour, 2 hours, 3 hours, 4 hours, 5 hours, 6 hours, 7 hours, 8 hours, 12 hours, 16 hours, 20 hours or 24 hours before or after, respectively, the administration of the NK cell engaging agent. Administration of the inhibitor of Src, JAK and/or mTOR signaling may be intermittently or continuously. In some aspects, administration of the inhibitor of Src, JAK and/or mTOR signaling is oral. In some aspects, administration of the inhibitor of Src, JAK and/or mTOR signaling is parenteral, particularly intravenous.

In some aspects, administration of the inhibitor of Src, JAK and/or mTOR signaling is at a dose sufficient to cause inhibition of an activity of the NK cell engaging agent. In some aspects, administration of the inhibitor of Src, JAK and/or mTOR signaling is at a dose insufficient to cause inhibition of another activity of the NK cell engaging agent. In some aspects, administration of the inhibitor of Src, JAK and/or mTOR signaling is at a dose sufficient to cause inhibition of a first activity of the NK cell engaging agent but insufficient to cause inhibition of a second activity of the NK cell engaging agent. In some of these aspects, said inhibition is a complete inhibition.

In some aspects, administration of the inhibitor of Src, JAK and/or mTOR signaling is at a dose sufficient to cause inhibition of cytokine secretion by immune cells, particularly NK cells (induced by the NK cell engaging agent). In some aspects, said immune cells are CD16⁺ immune cells. In some aspects, said cytokine is one or more cytokine selected from the group consisting of IL-6, IFN-γ, IL-8, TNF-α, IL-2, IL-12, IL-1β, MCP-1 and IL-10, particularly the group consisting of IL-6, IFN-γ, IL-8 and TNF-α. Immune cells may include various immune cell types, such as NK cells, macrophages, monocytes, T cells etc. In some aspects, said NK cells are CD16⁺ NK cells. In some aspects, said T cells are γδ T cells. In some aspects, said inhibition is a complete inhibition.

In some aspects, administration of the inhibitor of Src, JAK and/or mTOR signaling is at a dose insufficient to cause inhibition of the activation of NK cells (induced by the NK cell engaging agent). In some aspects, said inhibition is a complete inhibition.

In some aspects, administration of the inhibitor of Src, JAK and/or mTOR signaling is at a dose insufficient to cause inhibition of the cytotoxic activity of NK cells (induced by the NK cell engaging agent). In some aspects, said inhibition is a complete inhibition.

In some aspects, administration of the inhibitor of Src, JAK and/or mTOR signaling is at a dose sufficient to causes inhibition of cytokine secretion by NK cells (induced by the NK cell engaging agent) but insufficient to cause inhibition of the activation and/or the cytotoxic activity of NK cells (induced by the NK cell engaging agent). In some of these aspects, said inhibition is a complete inhibition.

In some aspects, administration of the inhibitor of Src, JAK and/or mTOR signaling is at a dose sufficient to cause reduction of the serum level of one of more cytokine in the individual. In some aspects, administration of the inhibitor of Src, JAK and/or mTOR signaling is at a dose sufficient to cause reduction of the secretion of one of more cytokine by immune cells, particularly NK cells, in the individual. In some aspects, said immune cells are CD16⁺ immune cells. In some aspects, said one or more cytokine is selected from the group consisting of IL-6, IFN-γ, IL-8, TNF-α, IL-2, IL-12, IL-1β, MCP-1 and IL-10, particularly the group consisting of IL-6, IFN-γ, IL-8 and TNF-α. Immune cells may include various immune cell types, such as NK cells, macrophages, monocytes, T cells etc. In some aspects, said T cells are γδ T cells.

In some aspects, administration of the inhibitor of Src, JAK and/or mTOR signaling is at a dose sufficient to cause inhibition of an adverse effect related to the administration of the NK cell engaging agent. In some aspects, administration of the inhibitor of Src, JAK and/or mTOR signaling is at a dose insufficient to cause inhibition of a desired effect related to the administration of the NK cell engaging agent. In some aspects, administration of the inhibitor of Src, JAK and/or mTOR signaling is at a dose sufficient to cause inhibition of an adverse effect related to the administration of the NK cell engaging agent but insufficient to cause inhibition of a desired effect related to the administration of the NK cell engaging agent. In some of these aspects, said inhibition is a complete inhibition. In some of these aspects, said inhibition is clinically meaningful and/or statistically significant.

In some aspects, administration of the inhibitor of Src, JAK and/or mTOR signaling is at an effective dose.

An “effective amount” or “effective dose” of an agent, e.g. a inhibitor of Src, JAK and/or mTOR signaling or a NK cell engaging agent, refers to an amount effective, at dosages and for periods of time necessary, to achieve the desired therapeutic or prophylactic result.

In some aspects, the administration of the inhibitor of Src, JAK and/or mTOR signaling is at a dose equaling a dose strength available for the inhibitor of Src, JAK and/or mTOR signaling. Typically, several dose strengths (i.e. dosage forms such as tablets or capsules with a specific amount of active ingredient) are available for a given inhibitor of Src, JAK and/or mTOR signaling. Dosing the inhibitor of Src, JAK and/or mTOR signaling at such (commercially) available dose strengths will be most convenient. For example, if the inhibitor of Src, JAK and/or mTOR signaling is dasatinib, it may preferably be administred at a dose of 20 mg, 50 mg, 70 mg, 80 mg, 100 mg or 140 mg, particularly 100 mg (administration preferably being oral administration). For example, if the inhibitor of Src, JAK and/or mTOR signaling is everolimus, it may preferably be administred at a dose of 2.5 mg, 5 mg, 7.5 mg or 10 mg (administration preferably being oral administration). For example, if the inhibitor of Src, JAK and/or mTOR signaling is sirolimus, it may preferably be administered at a dose of 0.5 mg, 1 mg or 2 mg (administration preferably being oral administration). For example, if the inhibitor of Src, JAK and/or mTOR signaling is ruxolitinib, it may preferably be administred at a dose of 5 mg, 10 mg, 15 mg, 20 mg or 25 mg (administration preferably being oral administration). If the inhibitor of Src, JAK and/or mTOR signaling is temsirolimus, it may be administred for example at a dose of 12.5 mg or 25 mg (administration preferably being intravenous administration, particularly using a solution of 25 mg/ml active ingredient).

In some aspects, administration of the inhibitor of Src, JAK and/or mTOR signaling is daily. In some aspects, administration of the inhibitor of Src, JAK and/or mTOR signaling is once daily. In some aspects, administration of the inhibitor of Src, JAK and/or mTOR signaling is once daily at a dose as mentioned hereinabove. In some aspects, administration of the inhibitor of Src, JAK and/or mTOR signaling is for the period of time during which the adverse effect persists (i.e. administration of the inhibitor of Src, JAK and/or mTOR signaling is from manifestation of the adverse effect until reduction or disappearance of the adverse effect). In some aspects, administration of the inhibitor of Src, JAK and/or mTOR signaling is stopped after the adverse effect is prevented or mitigated. In some aspects, administration of the inhibitor of Src, JAK and/or mTOR signaling is stopped after reduction or disappearance of the adverse effect. Said reduction particularly is clinically meaningful and/or statistically significant. In some aspects, administration of the inhibitor of Src, JAK and/or mTOR signaling is once, twice, three times, four times, five times, six times, seven times, eight times, nine times or ten times, particularly once, twice, three times, four times, five times, six times, seven times, eight times, nine times or ten times in the course of the treatment of the individual with the NK cell engaging agent. In some aspects, administration of the inhibitor of Src, JAK and/or mTOR signaling is for 1 day, 2 days, 3 days, 4 days, 5 days, 6 days, 7 days, 8 days, 9 days or 10 days. In some aspects, administration of the inhibitor of Src, JAK and/or mTOR signaling is once daily for 1 day, 2 days, 3 days, 4 days, 5 days, 6 days, 7 days, 8 days, 9 days or 10 days. The administration of the inhibitor of Src, JAK and/or mTOR signaling is generally associated with the administration of the NK cell engaging agent. In some aspects, administration of the inhibitor of Src, JAK and/or mTOR signaling is associated with the first administration of the NK cell engaging agent. Said first administration is particularly the first administration of the NK cell engaging agent in the course of the treatment of the individual with the NK cell engaging agent. In some aspects, administration of the inhibitor of Src, JAK and/or mTOR signaling is concurrent with the first administration of the NK cell engaging agent. In some aspects, administration of the inhibitor of Src, JAK and/or mTOR signaling is prior to the first administration of the NK cell engaging agent. In some aspects, administration of the inhibitor of Src, JAK and/or mTOR signaling is subsequent to the first administration of the NK cell engaging agent. In some aspects, administration of the inhibitor of Src, JAK and/or mTOR signaling is subsequent to the first administration of the NK cell engaging agent and prior to a second administration of the NK cell engaging agent. Where administration of the inhibitor of Src, JAK and/or mTOR signaling is prior or subsequent to the (first) administration of the NK cell engaging agent, such administration of the inhibitor of Src, JAK and/or mTOR signaling may be, for example, within about 1 hour, 2 hours, 3 hours, 4 hours, 5 hours, 6 hours, 7 hours, 8 hours, 12 hours, 16 hours, 20 hours, 24 hours, 48 hours or 72 hours before or after, respectively, the administration of the NK cell engaging agent.

In some aspects, the administration of the NK cell engaging agent is for a longer period of time than the administration of the inhibitor of Src, JAK and/or mTOR signaling. In some aspects, the administration of the NK cell engaging agent continues after the administration of the inhibitor of Src, JAK and/or mTOR signaling is stopped. In some aspects, the administration of the NK cell engaging agent is a single administration or a repeated administration. In the course of the treatment of the individual with the NK cell engaging agent, the NK cell engaging agent may be administered once or several times. For example, treatment of the individual with the NK cell engaging agent may comprise multiple treatment cycles which each comprise one or more administrations of the NK cell engaging agent. In some aspects, the administration of the NK cell engaging agent comprises a first and a second administration.

For use in the present invention, the NK cell engaging agent would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

In some aspects, the administration of the NK cell engaging agent is at an effective dose. For systemic administration, an effective dose can be estimated initially from in vitro assays, such as cell culture assays. A dose can then be formulated in animal models to achieve a circulating concentration range that includes the IC₅₀ as determined in cell culture. Such information can be used to more accurately determine useful doses in humans. Initial dosages can also be estimated from in vivo data, e.g., animal models, using techniques that are well known in the art. Dosage amount and interval may be adjusted individually to provide plasma levels of the NK cell engaging agent which are sufficient to maintain therapeutic effect. Usual patient dosages for administration by injection range from about 0.1 to 50 mg/kg/day, typically from about 0.5 to 1 mg/kg/day.

Therapeutically effective plasma levels may be achieved by administering multiple doses each day. Levels in plasma may be measured, for example, by HPLC. In specific aspects, in particular wherein the NK cell engaging agent is an effector-enhanced anti-CD20 antibody (e.g. obinutuzumab), the administration of the NK cell engaging agent is at a dose of about 100 mg to about 1000 mg. In some such aspects, the dose is 100 mg. In particular such aspects, the dose is 1000 mg.

An effective amount of the NK cell engaging agent may be administered for prevention or treatment of disease. The appropriate route of administration and dosage of the NK cell engaging agent may be determined based on the type of disease to be treated, the type of the NK cell engaging agent, the severity and course of the disease, the clinical condition of the individual, the individual's clinical history and response to the treatment, and the discretion of the attending physician. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

The NK cell engaging agent and the inhibitor of Src, JAK and/or mTOR signaling can be administered by any suitable route, and may be administered by the same route of administration or by different routes of administration. In some aspects, the administration of the NK cell engaging agent is parenteral, particularly intravenous.

In some aspects, the administration of the NK cell engaging agent is the first administration of the NK cell engaging agent to the individual, particularly the first administration of the NK cell engaging agent in the course of the treatment of the individual with the NK cell engaging agent.

In some aspects, (administration of) the NK cell engaging agent induces (i.e. causes or increases) the activation of NK cells. In some aspects, (administration of) the NK cell engaging agent induces cytotoxic activity of NK cells. In some aspects, (administration of) the NK cell engaging agent induces cytokine secretion by NK cells. In some aspects, said cytokine is one or more cytokine selected from the group consisting of IL-6, IFN-γ, IL-8, TNF-α, IL-2, IL-12, IL-1β, MCP-1 and IL-10, particularly the group consisting of IL-6, IFN-γ, IL-8 and TNF-α. In some aspects, said NK cells are CD16⁺ NK cells.

In some aspects, administration of the NK cell engaging agent results in activation of NK cells, particularly at the site of the cancer (e.g. within a solid tumor cancer). Said activation may comprise proliferation of NK cells, differentiation of NK cells, cytokine secretion by NK cells, cytotoxic effector molecule release from NK cells, cytotoxic activity of NK cells, and expression of activation markers by NK cells. In some aspects, the administration of the NK cell engaging agent results in an increase of NK cell numbers at the site of the cancer (e.g. within a solid tumor cancer).

By “NK cell engaging agent” is meant an immunotherapeutic agent that exerts its effect through the activity of NK cells, particularly CD16⁺ NK cells. Such activity of NK cells may include cellular response(s) of NK cells, particularly CD16⁺ NK cells, such as proliferation, differentiation, expression of activation markers, cytokine secretion, cytotoxic effector molecule release and/or cytotoxic activity.

An NK cell engaging agent may induce or enhance activity of NK cells through stimulation of CD16, particularly CD16a, on NK cells. Thus, in some aspects, the NK cell engaging agent is a CD16 binding agent. In such aspects, the NK cell engaging agent comprises an antigen binding moiety that binds to CD16, particularly to CD16a, such as an Fc region or an antigen binding domain of an antibody that binds to CD16.

CD16 (also known as Fcγ receptor III, FcγRIII) is a cell surface antigen expressed on certain immune cells. It exists both as a transmembrane form (CD16a, Fcγ receptor IIIa), which is expressed e.g. on NK cells and activated macrophages, and as a glycosylphosphatidyl-inositol (GPI)-anchored form (CD16b, FcγRIIIb) expressed on neutrophils. “CD16” as used herein refers particularly to CD16a, also known as Fcγ receptor IIIa (see UniProt accession no. P08637 [entry version 215] and SEQ ID NO: 11 for the human protein). Accordingly, the term “CD16 positive cells” or “CD16⁺ cells” refers to cells expressing CD16, particularly CD16a.

In particular aspects, the NK cell engaging agent comprises an Fc region. In some aspects, the NK cell engaging agent is an antibody comprising an Fc region, particularly an IgG antibody comprising an Fc region, most particularly an IgG₁ antibody comprising an Fc region. In some aspects, the Fc region comprised in the NK cell engaging agent is an IgG Fc region, particularly a human IgG Fc region. In some aspects, the Fc region comprised in the NK cell engaging agent is an IgG₁ Fc region, particularly a human IgG₁ Fc region.

The Fc region comprised in the NK cell engaging is capable of binding to CD16, i.e. the Fc region binds to CD16 (also referred to as a CD16-binding Fc region). Such Fc region will also be an effector-competent Fc region, i.e. an Fc region that is capable of inducing effector functions, in particular antibody-dependent cell-mediated cytotoxicity (ADCC).

The term “effector functions” refers to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B cell receptor), and B cell activation.

Antibody-dependent cellular cytotoxicity (ADCC) is an immune mechanism leading to the lysis of antibody-coated target cells by immune effector cells, particularly NK cells. As used herein, the term “increased ADCC” or “enhanced ADCC” is defined as either an increase in the number of target cells that are lysed in a given time, at a given concentration of antibody in the medium surrounding the target cells, by the mechanism of ADCC defined above, and/or a reduction in the concentration of antibody, in the medium surrounding the target cells, required to achieve the lysis of a given number of target cells in a given time, by the mechanism of ADCC. The increase in ADCC is relative to the ADCC mediated by the same antibody produced by the same type of host cells, using the same standard production, purification, formulation and storage methods (which are known to those skilled in the art), but that has not been engineered. For example the increase in ADCC mediated by an antibody produced by host cells engineered to have an altered pattern of glycosylation (e.g. to express the glycosyltransferase, GnTIII, or other glycosyltransferases) by the methods described herein, is relative to the ADCC mediated by the same antibody produced by the same type of non-engineered host cells.

Assays to assess ADCC activity of an antibody are known in the art. Examples of in vitro assays to assess ADCC activity of an antibody are described in U.S. Pat. No. 5,500,362; Hellstrom et al. Proc Natl Acad Sci USA 83, 7059-7063 (1986) and Hellstrom et al., Proc Natl Acad Sci USA 82, 1499-1502 (1985); U.S. Pat. No. 5,821,337; Bruggemann et al., J Exp Med 166, 1351-1361 (1987). Alternatively, non-radioactive assays methods may be employed (see, for example, ACTI™ non-radioactive cytotoxicity assay for flow cytometry (CellTechnology, Inc. Mountain View, Calif.); and CytoTox 96® non-radioactive cytotoxicity assay (Promega, Madison, Wisc.)). Useful effector cells for such assays include peripheral blood mononuclear cells (PBMC) and Natural Killer (NK) cells. Alternatively, or additionally, ADCC activity of the antibody may be assessed in vivo, e.g. in an animal model such as that disclosed in Clynes et al., Proc Natl Acad Sci USA 95, 652-656 (1998).

NK cell engaging agents as contemplated herein typically further comprise an antigen binding moiety that enables their binding to a target cell antigen on a target cell such as a tumor cell. Accordingly, in some aspects, the NK cell engaging agent binds to a target cell antigen. Such NK cell engaging agents exert effects on their target cell, such as lysis of the target cell, through the activity of NK cells.

A “target cell antigen” as used herein refers to an antigenic determinant presented on the surface of a target cell, for example a cell in a tumor such as a cancer cell or a cell of the tumor stroma (in that case a “tumor cell antigen”). Preferably, the target cell antigen is not CD16, and/or is expressed on a different cell than CD16. In some aspects, the target cell antigen is CD20, particularly human CD20.

As used herein, the term “antigenic determinant” is synonymous with “antigen” and “epitope”, and refers to a site (e.g. a contiguous stretch of amino acids or a conformational configuration made up of different regions of non-contiguous amino acids) on a polypeptide macromolecule to which an antigen binding moiety binds, forming an antigen binding moiety-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM).

As used herein, the term “antigen binding moiety” refers to a polypeptide molecule that binds, including specifically binds, to an antigenic determinant. In some aspects, an antigen binding moiety is able to direct the entity to which it is attached (e.g. a second antigen binding moiety) to a target site, for example to a specific type of tumor cell bearing the antigenic determinant. Antigen binding moieties include antibodies and fragments thereof as further defined herein. Particular antigen binding moieties include an antigen binding domain of an antibody, comprising an antibody heavy chain variable region and an antibody light chain variable region. In certain aspects, the antigen binding moieties may comprise antibody constant regions as further defined herein and known in the art. Useful heavy chain constant regions include any of the five isotypes: α, δ, ϵ, γ, or μ. Useful light chain constant regions include any of the two isotypes: κ and λ.

By “specific binding” is meant that the binding is selective for the antigen and can be discriminated from unwanted or non-specific interactions. The term “bind” or “binding” herein generally refers to “specific binding”. The ability of an antigen binding moiety to bind to a specific antigenic determinant can be measured either through an enzyme-linked immunosorbent assay (ELISA) or other techniques familiar to one of skill in the art, e.g. surface plasmon resonance (SPR) technique (analyzed e.g. on a BIAcore instrument) (Liljeblad et al., Glyco J 17, 323-329 (2000)), and traditional binding assays (Heeley, Endocr Res 28, 217-229 (2002)). In some aspects, the extent of binding of an antigen binding moiety to an unrelated protein is less than about 10% of the binding of the antigen binding moiety to the antigen as measured, e.g., by SPR. In certain aspects, an antigen binding moiety that binds to the antigen, or an antibody comprising that antigen binding moiety, has a dissociation constant (K_{D}) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹ M to 10⁻¹³ M).

In particular aspects, the NK cell engaging agent is capable of simultaneous binding to the antigenic determinant on the NK cell (e.g. CD16, particularly CD16a) and the antigenic determinant on the target cell (e.g. a target cell antigen such as CD20). In some aspects, the NK cell engaging agent is capable of crosslinking the NK cell and the target cell by simultaneous binding to CD16 and the target cell antigen. In some aspects, such simultaneous binding results in lysis of the target cell, particularly a target cell antigen (e.g. CD20)-expressing tumor cell. In some aspects, such simultaneous binding results in activation of the NK cell. In some aspects, such simultaneous binding results in a cellular response of the NK cell, selected from the group of: proliferation, differentiation, cytokine secretion, cytotoxic effector molecule release, cytotoxic activity, and expression of activation markers. In some aspects, binding of the NK cell engaging agent to CD16 without simultaneous binding to the target cell antigen does not result in NK cell activation. In some aspects, the NK cell engaging agent is capable of directing cytotoxic activity of an NK cell to a target cell.

Exemplary NK cell engaging agents include antibodies, particularly effector-enhanced antibodies, such as obinutuzumab, imgatuzumab, margetuximab, mogamulizumab and others. These exemplary NK cell engaging agents bind to CD16 (particularly CD16A) through an (engineered) Fc domain. Further exemplary NK cell engaging agents include antibodies that bind to CD16 (particularly CD16A) through an antigen binding domain of an antibody, particularly bi/multi-specific antibodies that bind to CD16 and a target cell antigen (e.g. tetravalent, bispecific antibodies based on the ROCK® (Redirected Opimized Cell Killing; Affimed) platform, binding to CD16A and a target cell antigen through antigen binding domains). NK cell engaging agents may also be trispecific/trifunctional antibodies binding to CD16 (particularly CD16A) and a second NK cell antigen, as well as a target cell antigen (e.g. antibodies based on the TriNKET™ (Tri-specific NK cell Engager Therapies; Dragonfly) platform, binding to CD16A (through an Fc domain), NKG2D and a target cell antigen (through antigen binding domains); or trifunctional NK cell engagers (NKCE; Innate Pharma), binding to CD16A (through an Fc domain), NKp46 and a target cell antigen (through antigen binding domains)).

The term “antibody” herein is used in the broadest sense and encompasses various antibody structures, including but not limited to monoclonal antibodies, polyclonal antibodies, multispecific antibodies (e.g. bispecific antibodies), and antibody fragments so long as they exhibit the desired antigen-binding activity.

The terms “full length antibody,” “intact antibody,” and “whole antibody” are used herein interchangeably to refer to an antibody having a structure substantially similar to a native antibody structure.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂, diabodies, linear antibodies, single-chain antibody molecules (e.g. scFv), and single-domain antibodies. For a review of certain antibody fragments, see Hudson et al., Nat Med 9, 129-134 (2003). For a review of scFv fragments, see e.g. Pltickthun, in The Pharmacology of Monoclonal Antibodies, vol. 113, Rosenburg and Moore eds., Springer-Verlag, New York, pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046. Diabodies are antibody fragments with two antigen-binding sites that may be bivalent or bispecific. See, for example, EP 404,097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). Single-domain antibodies are antibody fragments comprising all or a portion of the heavy chain variable domain or all or a portion of the light chain variable domain of an antibody. In certain aspects, a single-domain antibody is a human single-domain antibody (Domantis, Inc., Waltham, Mass.; see e.g. U.S. Pat. No. 6,248,516 B1). Antibody fragments can be made by various techniques, including but not limited to proteolytic digestion of an intact antibody as well as production by recombinant host cells (e.g. E. coli or phage), as described herein.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6t h ed., W.H. Freeman and Co., page 91 (2007). A single VH or VL domain may be sufficient to confer antigen-binding specificity. As used herein in connection with variable region sequences, “Kabat numbering” refers to the numbering system set forth by Kabat et al., Sequences of Proteins of Immunological Interest, 5th Ed. Public Health Service, National Institutes of Health, Bethesda, Md. (1991).

As used herein, the amino acid positions of all constant regions and domains of the heavy and light chain are numbered according to the Kabat numbering system described in Kabat, et al., Sequences of Proteins of Immunological Interest, 5th ed., Public Health Service, National Institutes of Health, Bethesda, Md. (1991), referred to as “numbering according to Kabat” or “Kabat numbering” herein. Specifically the Kabat numbering system (see pages 647-660 of Kabat, et al., Sequences of Proteins of Immunological Interest, 5th ed., Public Health Service, National Institutes of Health, Bethesda, Md. (1991)) is used for the light chain constant domain CL of kappa and lambda isotype and the Kabat EU index numbering system (see pages 661-723) is used for the heavy chain constant domains (CH1, Hinge, CH2 and CH3), which is herein further clarified by referring to “numbering according to Kabat EU index” in this case.

The term “hypervariable region” or “HVR”, as used herein, refers to each of the regions of an antibody variable domain which are hypervariable in sequence and which determine antigen binding specificity, for example “complementarity determining regions” (“CDRs”). Generally, antibodies comprise six CDRs; three in the VH (HCDR1, HCDR2, HCDR3), and three in the VL (LCDR1, LCDR2, LCDR3). Exemplary CDRs herein include:

(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, Md. (1991)); and
(c) antigen contacts occurring at amino acid residues 27c-36 (L1), 46-55 (L2), 89-96 (L3), 30-35b (HO, 47-58 (H2), and 93-101 (H3) (MacCallum et al. J. Mol. Biol. 262: 732-745 (1996)).

Unless otherwise indicated, the CDRs are determined according to Kabat et al., supra. One of skill in the art will understand that the CDR designations can also be determined according to Chothia, supra, McCallum, supra, or any other scientifically accepted nomenclature system.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following order in VH (or VL): FR1-H1(L1)-FR2-H2(L2)-FR3 -H3 (L3)-FR4.

The “class” of an antibody or immunoglobulin refers to the type of constant domain or constant region possessed by its heavy chain. There are five maj or classes of antibodies: IgA, IgD, IgE, IgG, and IgM, and several of these may be further divided into subclasses (isotypes), e.g., IgG₁, IgG₂, IgG₃, IgG₄, TgA₁, and IgA₂. The heavy chain constant domains that correspond to the different classes of immunoglobulins are called α, δ, ϵ, γ, and μ, respectively.

The term “immunoglobulin molecule” refers to a protein having the structure of a naturally occurring antibody. For example, immunoglobulins of the IgG class are heterotetrameric glycoproteins of about 150,000 daltons, composed of two light chains and two heavy chains that are disulfide-bonded. From N- to C-terminus, each heavy chain has a variable domain (VH), also called a variable heavy domain or a heavy chain variable region, followed by three constant domains (CH1, CH2, and CH3), also called a heavy chain constant region. Similarly, from N- to C-terminus, each light chain has a variable domain (VL), also called a variable light domain or a light chain variable region, followed by a constant light (CL) domain, also called a light chain constant region. The heavy chain of an immunoglobulin may be assigned to one of five types, called α (IgA), δ (IgD), ϵ (IgE), γ (IgG), or μ (IgM), some of which may be further divided into subtypes, e.g. γ₁ (IgG₁), γ₂ (IgG₂), γ₃ (IgG₃), γ₄ (IgG₄), α₁ (IgA₁) and α₂ (IgA₂). The light chain of an immunoglobulin may be assigned to one of two types, called kappa (κ) and lambda (λ), based on the amino acid sequence of its constant domain. An immunoglobulin essentially consists of two Fab molecules and an Fc domain, linked via the immunoglobulin hinge region.

The term “Fc domain” or “Fc region” herein is used to define a C-terminal region of an immunoglobulin heavy chain that contains at least a portion of the constant region. The term includes native sequence Fc regions and variant Fc regions. Although the boundaries of the Fc region of an IgG heavy chain might vary slightly, the human IgG heavy chain Fc region is usually defined to extend from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447, numbering according to Kabat EU index). Therefore, the C-terminal lysine (Lys447), or the C-terminal glycine (Gly446) and lysine (K447), of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991 (see also above).

In particularly preferred aspects, the Fc domain herein is a human IgGi Fc domain. An exemplary sequence of a human IgGi Fc region is given in SEQ ID NO: 1.

In particular aspects of the present invention, the NK cell engaging agent is an antibody, particularly an effector-enhanced antibody.

Antibodies with enhanced effector function, particularly enhanced ADCC capacity, are an emerging species in the field of cancer therapy. It has been recognized that the effector functions of an antibody, which are mediated by its Fc region, are an important mechanism of action in antibody-based cancer therapy. Of particular importance in this context is antibody-dependent cellular cytotoxicity (ADCC), the destruction of antibody-coated target cells (e.g. tumor cells) by NK (natural killer cells) and other immune effector cells, which is triggered when antibody bound to the surface of a cell interacts with activating Fc receptors on the effector cell.

Enhancing the ADCC activity of therapeutic antibodies has therefore become of great interest and various methods for ADCC enhancement have been described. For example, Shields et al. (J Biol Chem 9(2), 6591-6604 (2001)) showed that amino acid substitutions at positions 298, 333, and/or 334 of the Fc region (EU numbering of residues) improve ADCC. Alternatively, increased Fc receptor binding and effector function can be obtained by altering the glycosylation of an antibody. IgG1 type antibodies, the most commonly used antibodies in cancer immunotherapy, have a conserved N-linked glycosylation site at Asn 297 in each CH2 domain of the Fc region. The two complex biantennary oligosaccharides attached to Asn 297 are buried between the CH2 domains, forming extensive contacts with the polypeptide backbone, and their presence is essential for the antibody to mediate effector functions including ADCC (Lifely et al., Glycobiology 5, 813-822 (1995); Jefferis et al., Immunol Rev 163, 59-76 (1998); Wright and Morrison, Trends Biotechnol 15, 26-32 (1997)). Umaña et al. (Nat Biotechnol 17, 176-180 (1999) and U.S. Pat. No. 6,602,684 (WO 99/54342), the contents of which are hereby incorporated by reference in their entirety) showed that overexpression of β(1,4)-N-acetylglucosaminyltransferase III (GnTIII), a glycosyltransferase catalyzing the formation of bisected oligosaccharides, in Chinese hamster ovary (CHO) cells significantly increases the in vitro ADCC activity of antibodies produced in those cells. Overexpression of GnTIII in production cell lines leads to antibodies enriched in bisected oligosaccharides, which are generally also non-fucosylated and of the hybrid type. If in addition to GnTIII, mannosidase II (ManII) is overexpressed in production cell lines, antibodies enriched in bisected, non-fucosylated oligosaccharides of the complex type are obtained (Ferrara et al., Biotechn Bioeng 93, 851-861 (2006)). Both types of antibodies show strongly increased ADCC, as compared to antibodies with unmodified glycans, but only antibodies in which the majority of the N-glycans are of the complex type are able to induce significant complement-dependent cytotoxicity (Ferrara et al., Biotechn Bioeng 93, 851-861 (2006)). The elimination of fucose from the innermost N-acetylglucosamine residue of the oligosaccharide core appears to be the critical factor for the increase of ADCC activity (Shinkawa et al., J Biol Chem 278, 3466-3473 (2003)). Therefore, further methods for producing antibodies with reduced fucosylation were developed, including e.g. expression in α(1,6)-fucosyltransferase deficient host cells (Yamane-Ohnuki et al., Biotech Bioeng 87, 614-622 (2004); Niwa et al., J Immunol Methods 306, 151-160 (2006)).

Several effector-enhanced antibodies, including the glycoengineered anti-EGFR antibody imgatuzumab, as well as the glycoengineered anti-CD20 antibody obinutuzumab have shown promising results in the clinic. Obinutuzumab is marketed under the trade name Gazyva®/Gazyvaro® for the treatment of certain forms of follicular lymphoma (FL) and chronic lymphocytic leukemia (CLL).

An “effector-enhanced antibody” as defined herein for the various aspects of the present invention is an antibody engineered to have increased effector function, particularly increased ADCC activity and/or increased CD16 (particularly CD16a) binding, as compared to a corresponding non-engineered antibody. In some aspects, the effector-enhanced antibody has at least 2-fold, at least 10-fold or even at least 100-fold increased effector function, compared to a corresponding non-engineered antibody. In particular aspects, the increased effector function is increased binding to CD16, particularly CD16a, most particularly human CD16a. In some such aspects, the binding affinity to CD16 is increased at least 2-fold, particularly at least 10-fold, compared to the binding affinity of a corresponding non-engineered antibody. In some aspects, the increased effector function is increased ADCC. In some such aspects, the ADCC is increased at least 2-fold, particularly at least 10-fold, compared to the ADCC mediated by a corresponding non-engineered antibody. In some aspects, the increased effector function is increased binding to an activating Fc receptor and increased ADCC.

Increased effector function may result e.g. from glycoengineering of the Fc region or the introduction of amino acid mutations in the Fc region of the antibody. In some aspects, the effector-enhanced antibody is engineered by introduction of one or more amino acid mutations in the Fc region. In some such aspects, the amino acid mutations are amino acid substitutions. In specific such aspects, the amino acid substitutions are at positions 298, 333, and/or 334 of the Fc region (EU numbering of residues). Further suitable amino acid mutations are described e.g. in Shields et al., J Biol Chem 9(2), 6591-6604 (2001); U.S. Pat. No. 6,737,056; WO 2004/063351 and WO 2004/099249. Mutant Fc regions can be prepared by amino acid deletion, substitution, insertion or modification using genetic or chemical methods well known in the art. Genetic methods may include site-specific mutagenesis of the encoding DNA sequence, PCR, gene synthesis, and the like. The correct nucleotide changes can be verified for example by sequencing.

In some aspects, the effector-enhanced antibody is engineered by modification of the glycosylation in the Fc region. In specific aspects, the effector-enhanced antibody is engineered to have an increased proportion of non-fucosylated oligosaccharides in the Fc region as compared to a non-engineered antibody. An increased proportion of non-fucosylated oligosaccharides in the Fc region of an antibody results in the antibody having increased effector function, in particular increased ADCC.

In particular aspects, the effector-enhanced antibody is a glycoengineered antibody comprising an increased proportion of non-fucosylated oligosaccharides in its Fc region, compared to a non-glycoengineered antibody. In some such aspects, the antibody is produced in a host cell engineered to have increased β(1,4)-N-acetylglucosaminyltransferase III (GnTIII) activity, compared to a non-engineered host cell. In more specific aspects, the host cell additionally is engineered to have increased α-mannosidase II (ManII) activity, compared to a non-engineered host cell. A host cell may be engineered to have increased β(1,4)-N-acetylglucosaminyltransferase III (GnTIII) activity by overexpression of one or more polypeptides having β(1,4)-N-acetylglucosaminyltransferase III (GnTIII) activity. Likewise, a host cell may be engineered to have increased α-mannosidase II (ManII) activity by overexpression of one or more polypeptides having a-mannosidase II (ManII) activity. This glycoengineering methodology has been described in greater detail in Umaria et al., Nat Biotechnol 17, 176-180 (1999); Ferrara et al., Biotechn Bioeng 93, 851-861 (2006); WO 99/54342; WO 2004/065540, WO 03/011878, the entire content of each of which is incorporated herein by reference in its entirety.

In alternative aspects, the effector-enhanced antibody is a glycoengineered antibody comprising an increased proportion of non-fucosylated oligosaccharides in its Fc region, compared to a non-glycoengineered antibody, wherein the antibody is produced in a host cell having decreased α(1,6)-fucosyltransferase activity. A host cell having decreased α(1,6)-fucosyltransferase activity may be a cell in which the α(1,6)-fucosyltransferase gene has been disrupted or otherwise deactivated, e.g. knocked out (see Yamane-Ohnuki et al., Biotech Bioeng 87, 614 (2004); Kanda et al., Biotechnol Bioeng, 94(4), 680-688 (2006); Niwa et al., J Immunol Methods 306, 151-160 (2006)).

Other examples of cell lines capable of producing defucosylated antibodies include Lec13 CHO cells deficient in protein fucosylation (Ripka et al., Arch Biochem Biophys 249, 533-545 (1986); US Pat. Appl. No. US 2003/0157108; and WO 2004/056312, especially at Example 11). The antibodies useful in the present invention can alternatively be glycoengineered to have reduced fucose residues in the Fc region according to the techniques disclosed in EP 1 176 195 A1, WO 03/084570, WO 03/085119 and U.S. Pat. Appl. Pub. Nos. 2003/0115614, 2004/093621, 2004/110282, 2004/110704, 2004/132140, U.S. Pat. No. 6,946,292 (Kyowa), e.g. by reducing or abolishing the activity of a GDP-fucose transporter protein in the host cells used for antibody production.

Glycoengineered antibodies useful in the invention may also be produced in expression systems that produce modified glycoproteins, such as those taught in WO 03/056914 (GlycoFi, Inc.) or in WO 2004/057002 and WO 2004/024927 (Greenovation).

In some aspects, the effector-enhanced antibody is engineered to have an increased proportion of non-fucosylated oligosaccharides in the Fc region as compared to a non-engineered antibody. In some aspects, at least about 20%, about 40%, about 60% or about 80%, preferably at least about 40%, of the N-linked oligosaccharides in the Fc region of the effector-enhanced antibody are non-fucosylated. In some aspects, between about 40% and about 80% of the N-linked oligosaccharides in the Fc region of the effector-enhanced antibody are non-fucosylated. The non-fucosylated oligosaccharides may be of the hybrid or complex type.

In some aspects, the effector-enhanced antibody is engineered to have an increased proportion of bisected oligosaccharides in the Fc region as compared to a non-engineered antibody. In some aspects, at least about 20%, about 40%, about 60% or about 80%, preferably at least about 40%, of the N-linked oligosaccharides in the Fc region of the effector-enhanced antibody are bisected. In some aspects, between about 40% and about 80% of the N-linked oligosaccharides in the Fc region of the effector-enhanced antibody are bisected. The bisected oligosaccharides may be of the hybrid or complex type.

In some aspects, the effector-enhanced antibody is engineered to have an increased proportion of bisected, non-fucosylated oligosaccharides in the Fc region, as compared to a non-engineered antibody. In some aspects, at least about 20%, about 40%, about 60% or about 80%, preferably at least about 40%, of the N-linked oligosaccharides in the Fc region of the effector-enhanced antibody are bisected, non-fucosylated. In some aspects, between about 40% and about 80% of the N-linked oligosaccharides in the Fc region of the effector-enhanced antibody are bisected, non-fucosylated. The bisected, non-fucosylated oligosaccharides may be of the hybrid or complex type.

In some aspects, the effector-enhanced antibody is an antibody having at least about 20%, about 40%, about 60% or about 80% non-fucosylated oligosaccharides in its Fc region. In some aspects, the effector-enhanced antibody is an antibody having at least about 40% non-fucosylated oligosaccharides in its Fc region. In some aspects, the effector-enhanced antibody is an antibody having at least about 20%, about 40%, about 60% or about 80% bisected oligosaccharides in its Fc region. In some aspects, the effector-enhanced antibody is an antibody having at least about 40% bisected, non-fucosylated oligosaccharides in its Fc region.

The oligosaccharide structures in the antibody Fc region can be analysed by methods well known in the art, e.g. by MALDI TOF mass spectrometry as described in Umaria et al., Nat Biotechnol 17, 176-180 (1999) or Ferrara et al., Biotechn Bioeng 93, 851-861 (2006). The percentage of non-fucosylated oligosaccharides is the amount of oligosaccharides lacking fucose residues, relative to all oligosaccharides attached to Asn 297 (e. g. complex, hybrid and high mannose structures) and identified in an N-glycosidase F treated sample by MALDI TOF MS. Asn 297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. The percentage of bisected, or bisected non-fucosylated, oligosaccharides is determined analogously.

As used herein, the terms “engineer, engineered, engineering” are considered to include any manipulation of the peptide backbone or the post-translational modifications of a naturally occurring or recombinant polypeptide or fragment thereof. Engineering includes modifications of the amino acid sequence, of the glycosylation pattern, or of the side chain group of individual amino acids, as well as combinations of these approaches. “Engineering”, particularly with the prefix “glyco-”, as well as the term “glycosylation engineering” includes metabolic engineering of the glycosylation machinery of a cell, including genetic manipulations of the oligosaccharide synthesis pathways to achieve altered glycosylation of glycoproteins expressed in cells. Furthermore, glycosylation engineering includes the effects of mutations and cell environment on glycosylation. In some aspects, the glycosylation engineering is an alteration in glycosyltransferase activity. Glycosyltransferases include β(1,4)-N-acetylglucosaminyltransferase III (GnTIII), β(1,4)-galactosyltransferase (GalT), β(1,2)-N-acetylglucosaminyltransferase I (GnTI), β(1,2)-N-acetylglucosaminyltransferase II (GnTII) and α(1,6)-fucosyltransferase. In particular aspects, the engineering results in altered glucosaminyltransferase activity and/or fucosyltransferase activity (e.g. as described hereinabove).

“Increased binding”, for example increased binding to CD16, refers to a increase in affinity for the respective interaction, as measured for example by SPR.

“Affinity” refers to the strength of the sum total of non-covalent interactions between a single binding site of a molecule (e.g., a receptor) and its binding partner (e.g., a ligand). Unless indicated otherwise, as used herein, “binding affinity” refers to intrinsic binding affinity which reflects a 1:1 interaction between members of a binding pair (e.g., an antigen binding moiety and an antigen, or a receptor and its ligand). The affinity of a molecule X for its partner Y can generally be represented by the dissociation constant (K_{D}), which is the ratio of dissociation and association rate constants (k_{off} and kₘ, respectively). Thus, equivalent affinities may comprise different rate constants, as long as the ratio of the rate constants remains the same. Affinity can be measured by well established methods known in the art, including those described herein. A particular method for measuring affinity is Surface Plasmon Resonance (SPR).

Binding affinity to CD16 can be easily determined e.g. by Surface Plasmon Resonance (SPR) using standard instrumentation such as a BIAcore instrument (GE Healthcare), and CD16 such as may be obtained by recombinant expression. In some aspects, binding affinity to CD16 (particularly CD16a) is measured by surface plasmon resonance at 25° C.

In some aspects, the effector-enhanced antibody is a full-length antibody. In some aspects, the effector-enhanced antibody is an IgG antibody. In particular aspects, the effector-enhanced antibody is an IgGi antibody. The effector-enhanced antibody comprises an Fc region, particularly an IgG Fc region, more particularly an IgGi Fc region. In some aspects, the Fc region is a human Fc region, particularly a human IgG Fc region, more particularly a human IgGi Fc region.

The effector-enhanced antibody binds to a target cell antigen on a target cell such as a tumor cell.

In some aspects, the effector-enhanced antibody binds to CD20, particularly human CD20 (i.e. the effector-enhanced antibody is an anti-CD20, particularly anti-human CD20, antibody).

“CD20”, also known as “B-lymphocyte antigen B1”, refers to any native CD20 from any vertebrate source, including mammals such as primates (e.g. humans), non-human primates (e.g. cynomolgus monkeys) and rodents (e.g. mice and rats), unless otherwise indicated. The term encompasses “full-length,” unprocessed CD20 as well as any form of CD20 that results from processing in the cell. The term also encompasses naturally occurring variants of CD20, e.g., splice variants or allelic variants. In some aspects, CD20 is human CD20. Human CD20 is described in UniProt (www.uniprot.org) accession no. P11836 (entry version 202), and an amino acid sequence of human CD20 is also shown in SEQ ID NO: 10.

In some aspects, the NK cell engaging agent is a effector-enhanced anti-CD20 antibody. In some aspects, the anti-CD20 antibody is an IgG antibody, particularly an IgGi antibody. In some aspects, the anti-CD20 antibody is a full length antibody. In some aspects, the anti-CD20 antibody comprises an Fc region, particularly an IgG Fc region, more particularly an IgG1 Fc region. In some aspects, the anti-CD20 antibody comprises a human Fc region, particularly a human IgG Fc region, more particularly a human IgG₁ Fc region. In some aspects, the anti-CD20 antibody is engineered to have an increased proportion of non-fucosylated oligosaccharides in the Fc region as compared to a non-engineered antibody. In some aspects, at least about 40% of the N-linked oligosaccharides in the Fc region of the anti-CD20 antibody are non-fucosylated.

In some aspects, the anti-CD20 antibody comprises a heavy chain variable region comprising the heavy chain CDR (HCDR) 1 of SEQ ID NO: 2, the HCDR2 of SEQ ID NO: 3, and the HCDR3 of SEQ ID NO: 4; and a light chain variable region comprising the light chain CDR (LCDR) 1 of SEQ ID NO: 5, the LCDR2 of SEQ ID NO: 6 and the LCDR3 of SEQ ID NO: 7. In some aspects, the anti-CD20 antibody comprises a heavy chain variable region sequence that is at least about 95%, 96%, 97%, 98%, 99% or 100% identical to the amino acid sequence of SEQ ID NO: 8 and/or a light chain variable region sequence that is at least about 95%, 96%, 97%, 98%, 99% or 100% identical to the amino acid sequence of SEQ ID NO: 9. In some aspects, the anti-CD20 antibody comprises the heavy chain variable region sequence of SEQ ID NO: 8 and/or the light chain variable region sequence of SEQ ID NO: 9.

In particular aspects, the anti-CD20 antibody is obinutuzumab (recommended INN, WHO Drug Information, Vol. 26, No. 4, 2012, p. 453). As used herein, obinutuzumab is synonymous for GA101. The tradename is GAZYVA® or GAZYVARO®.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, Clustal W, Megalign (DNASTAR) software or the FASTA program package. Those skilled in the art can determine appropriate parameters for aligning sequences, including any algorithms needed to achieve maximal alignment over the full length of the sequences being compared. For purposes herein, however, % amino acid sequence identity values are generated using the ggsearch program of the FASTA package version 36.3.8c or later with a BLOSUM50 comparison matrix. The FASTA program package was authored by W. R. Pearson and D. J. Lipman (1988), “Improved Tools for Biological Sequence Analysis”, PNAS 85:2444-2448; W. R. Pearson (1996) “Effective protein sequence comparison” Meth. Enzymol. 266:227- 258; and Pearson et. al. (1997) Genomics 46:24-36, and is publicly available from http://fasta.bioch.virginia.edu/fasta_www2/fasta_down.shtml. Alternatively, a public server accessible at http://fasta.bioch.virginia.edu/fasta_www2/index.cgi can be used to compare the sequences, using the ggsearch (global protein:protein) program and default options (BLOSUM50; open: −10; ext: −2; Ktup=2) to ensure a global, rather than local, alignment is performed. Percent amino acid identity is given in the output alignment header.

In some aspects, the disease (to be treated by the NK cell engaging agent) is cancer.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

The term “cancer” refers to the physiological condition in mammals that is typically characterized by unregulated cell proliferation. Examples of cancer include but are not limited to, carcinoma, lymphoma, blastoma, sarcoma and leukemia. More non-limiting examples of cancers include haematological cancer such as leukemia, bladder cancer, brain cancer, head and neck cancer, pancreatic cancer, biliary cancer, thyroid cancer, lung cancer, breast cancer, ovarian cancer, uterine cancer, cervical cancer, endometrial cancer, esophageal cancer, colon cancer, colorectal cancer, rectal cancer, gastric cancer, prostate cancer, skin cancer, squamous cell carcinoma, sarcoma, bone cancer, and kidney cancer. Other cell proliferation disorders include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous system (central and peripheral), lymphatic system, pelvic, skin, soft tissue, spleen, thoracic region, and urogenital system. Also included are pre-cancerous conditions or lesions and cancer metastases.

In some aspects, the cancer is a cancer expressing the target cell antigen of the NK cell engaging agent (e.g. the effector-enhanced antibody).

In some aspects, the cancer is a CD20-expressing cancer (in particular in aspects, wherein the target cell antigen of the NK cell engaging agent, e.g. effector-enhanced antibody, is CD20). By “CD20-positive cancer” or “CD20-expressing cancer” is meant a cancer characterized by expression or overexpression of CD20 in cancer cells. The expression of CD20 may be determined for example by quantitative real-time PCR (measuring CD20 mRNA levels), immunohistochemistry (IHC) or western blot assays. In some aspects, the cancer expresses CD20.

In some aspects, the cancer expresses CD20 in at least 20%, preferably at least 50% or at least 80% of tumor cells as determined by immunohistochemistry (IHC) using an antibody specific for CD20.

In some aspects, the cancer is a B-cell cancer, particularly a CD20-positive B-cell cancer. In some aspects, the cancer is selected from the group consisting of Non-Hodgkin lymphoma (NHL), acute lymphocytic leukemia (ALL), chronic lymphocytic leukemia (CLL), diffuse large B-cell lymphoma (DLBCL), follicular lymphoma (FL), mantle-cell lymphoma (MCL), marginal zone lymphoma (MZL), Multiple myeloma (MM) or Hodgkin lymphoma (HL). In particular aspects, the cancer is selected from the group consisting of Non-Hodgkin lymphoma (NHL), acute lymphocytic leukemia (ALL), chronic lymphocytic leukemia (CLL), diffuse large B-cell lymphoma (DLBCL), follicular lymphoma (FL), mantle-cell lymphoma (MCL) and marginal zone lymphoma (MZL). In more particular aspects, the cancer is FL. In some aspects, the cancer is CLL.

In some aspects, the cancer is treatable by the NK cell engaging agent. In some aspects, the NK cell engaging agent is indicated for the treatment of the cancer.

An “individual” or “subject” herein is a mammal. Mammals include, but are not limited to, domesticated animals (e.g. cows, sheep, cats, dogs, and horses), primates (e.g. humans and non-human primates such as monkeys), rabbits, and rodents (e.g. mice and rats). In certain aspects, the individual or subject is a human. In some aspects, the individual has a disease, particularly a disease treatable or to be treated by the NK cell engaging agent. In some aspects, the individual has cancer, particularly a cancer treatable or to be treated by the NK cell engaging agent. In particular, an individual herein is any single human subject eligible for treatment who is experiencing or has experienced one or more signs, symptoms, or other indicators of cancer. In some aspects, the individual has cancer or has been diagnosed with cancer, in particular any of the cancers described hereinabove. In some aspects, the individual has locally advanced or metastatic cancer or has been diagnosed with locally advanced or metastatic cancer. The individual may have been previously treated with an NK cell engaging agent (e.g. an effector-enhanced antibody) or another drug, or not so treated. In particular aspects, the patient has not been previously treated with an NKcell engaging agent (e.g. an effector-enhanced antibody). The patient may have been treated with a therapy comprising one or more drugs other than an NK cell engaging agent (e.g. other than an effector-enhanced antibody) before the NK cell engaging agent therapy is commenced.

In some aspects, the individual has an elevated serum level of one of more cytokine. In some aspects, said elevated serum level is related to the administration of the NK cell engaging agent to the individual. Said elevated serum level is in particular as compared to the serum level in a healthy individual, and/or the serum level in an individual (including the same individual) without administration of the NK cell engaging agent (i.e. in such case the serum level is elevated as compared to the serum level without administration of the NK cell engaging agent). In some aspects, said one or more cytokine is selected from the group consisting of IL-6, IFN-γ, IL-8, TNF-α, IL-2, IL-12, IL-1β, MCP-1 and IL-10, particularly the group consisting of IL-6, IFN-γ, IL-8 and TNF-α.

A cytokine according to any of the aspects of the invention may be one or more cytokine selected from the group consisting of interleukin (IL)-6, interferon (IFN)-γ, IL-8, tumor necrosis factor (TNF)-α, IL-2, monocyte chemoattractant protein (MCP)-1, IL-12, IL-1β and IL-10. In some aspects, the cytokine is one or more cytokine selected from the group consisting of IL-6, IFN-γ, IL-8 and TNF-α, IL-2 and MCP-1. In some aspects, the cytokine is one or more cytokine selected from the group consisting of IL-6, IFN-γ, IL-8, TNF-α and MCP-1. In some aspects, the cytokine is one or more cytokine selected from the group consisting of IL-6, IFN-γ, IL-8 and TNF-α. In some aspects, the cytokine is IL-6. In some aspects, the cytokine is IFN-γ. In some aspects, the cytokine is IL-8. In some aspects, the cytokine is TNF-α. In some aspects, the cytokine is MCP-1.

In some aspects, the cytokine is IL-1β. In some aspects, the cytokine is IL-10. In some apsects, the cytokine is IL-12. In some aspects, the cytokine is IL-2.

Preferably, a NK cell according to any of the aspects of the invention is a CD16⁺ NK cell.

In some aspects, the treatment with or administration of the NK cell engaging agent may result in a response in the individual. In some aspects, the response may be a complete response. In some aspects, the response may be a sustained response after cessation of the treatment. In some aspects, the response may be a complete response that is sustained after cessation of the treatment. In other aspects, the response may be a partial response. In some aspects, the response may be a partial response that is sustained after cessation of the treatment. In some aspects, the treatment with or administration of the NK cell engaging agent and the inhibitor of Src, JAK and/or mTOR signaling may improve the response as compared to treatment with or administration of the NK cell engaging agent alone (i.e. without the inhibitor of Src, JAK and/or mTOR signaling). In some aspects, the treatment or administration of the NK cell engaging agent and the inhibitor of Src, JAK and/or mTOR signaling may increase response rates in a patient population, as compared to a corresponding patient population treated with the NK cell engaging agent alone (i.e. without the inhibitor of Src, JAK and/or mTOR signaling).

The NK cell engaging agent may be used alone or together with other agents in a therapy. For instance, a NK cell engaging agent may be co-administered with at least one additional therapeutic agent. In certain aspects, an additional therapeutic agent is an anti-cancer agent, e.g. a chemotherapeutic agent, an inhibitor of tumor cell proliferation, or an activator of tumor cell apoptosis. In specific aspects, in particular wherein the NK cell engaging agent is an effector-enhanced anti-CD20 antibody (e.g. obinutuzumab), an additional therapeutic agent is selected from cyclophosphamide, doxorubicin, vincristine, prednisone or prednisolone, chlorambucil or bendamustine. In some such aspects, an additional therapeutic agent is a combination of chemotherapeutic agents, particularly a combination of cyclophosphamide, doxorubicin, vincristine, and prednisone or prednisolone (CHOP), or a combination of cyclophosphamide, vincristine, and prednisone or prednisolone (CVP).

The inhibitor of Src, JAK and/or mTOR signaling may be used alone or together with one or more other agents for the prevention of mitigation of an adverse effect, particularly CRS, related to the administration of the NK cell engaging agent. The inhibitor of Src, JAK and/or mTOR signaling may for example be used together with an IL-6R antagonist (e.g. tocilizumab), a steroid (e.g. a corticosteroid such as methylprednisolone or dexamethasone) or a TNF-α antagonist (e.g. etanercept).

Amino Acid Sequences

| | | SEQ |
|---|---|---|
| | Sequence | ID NO |
| hIgG1 Fc | DKTHTCPPCPAPELLGGPSVFLFPPKPKDTLMISRTPEVTCV | 1 |
| region | VVDVSHEDPEVKFNWYVDGVEVHNAKTKPREEQYNSTYR | |
| | VVSVLTVLHQDWLNGKEYKCKVSNKALPAPIEKTISKAKG | |
| | QPREPQVYTLPPSRDELTKNQVSLTCLVKGFYPSDIAVEWE | |
| | SNGQPENNYKTTPPVLDSDGSFFLYSKLTVDKSRWQQGNV | |
| | FSCSVMHEALHNHYTQKSLSLSP | |
| CD20 HCDR1 | YSWIN | 2 |
| CD20 HCDR2 | RIFPGDGDTDYNGKFKG | 3 |
| CD20 HCDR3 | NVFDGYWLVY | 4 |
| CD20 LCDR1 | RSSKSLLHSNGITYLY | 5 |
| CD20 LCDR2 | QMSNLVS | 6 |
| CD20 LCDR3 | AQNLELPYT | 7 |
| CD20 VH | QVQLVQSGAEVKKPGSSVKVSCKASGYAFSYSWINWVRQ | 8 |
| | APGQGLEWMGRIFPGDGDTDYNGKFKGRVTITADKSTSTA | |
| | YMELSSLRSEDTAVYYCARNVFDGYWLVYWGQGTLVTVS | |
| | S | |
| CD20 VL | DIVMTQTPLSLPVTPGEPASISCRSSKSLLHSNGITYLYWYL | 9 |
| | QKPGQSPQLLIYQMSNLVSGVPDRFSGSGSGTDFTLKISRVE | |
| | AEDVGVYYCAQNLELPYTFGGGTKVEIKRTV | |
| Human CD20 | MTTPRNSVNGTFPAEPMKGPIAMQSGPKPLFRRMSSLVGPT | 10 |
| | QSFFMRESKTLGAVQIMNGLFHIALGGLLMIPAGIYAPICVT | |
| | VWYPLWGGIMYIISGSLLAATEKNSRKCLVKGKMIMNSLS | |
| | LFAAISGMILSIMDILNIKISHFLKMESLNFIRAHTPYINIYNC | |
| | EPANPSEKNSPSTQYCYSIQSLFLGILSVMLIFAFFQELVIAGI | |
| | VENEWKRTCSRPKSNIVLLSAEEKKEQTIEIKEEVVGLTETS | |
| | SQPKNEEDIEIIPIQEEEEEETETNFPEPPQDQESSPIENDSSP | |
| Human | MWQLLLPTALLLLVSAGMRTEDLPKAVVFLEPQWYRVLE | 11 |
| CD16A | KDSVTLKCQGAYSPEDNSTQWFHNESLISSQASSYFIDAAT | |
| | VDDSGEYRCQTNLSTLSDPVQLEVHIGWLLLQAPRWVFKE | |
| | EDPIHLRCHSWKNTALHKVTYLQNGKGRKYFHHNSDFYIP | |
| | KATLKDSGSYFCRGLFGSKNVSSETVNITITQGLAVSTISSFF | |
| | PPGYQVSFCLVMVLLFAVDTGLYFSVKTNIRSSTRDWKDH | |
| | KFKWRKDPQDK | |

### BRIEF DESCRIPTION OF THE DRAWINGS

FIG. 1. Effect of 100 nM ruxolitinib (ruxo), 100 nM sirolimus (siro), 100 nM dasatinib (dasa) on obinutuzumab-induced CD19⁺ B cell depletion in a whole blood assay. Fresh whole blood was incubated with 100, 10, 1 and 0.1 μg/mL obinutuzumab or PGLALA IgG in the presence and absence of 100 nM ruxolitinib, 100 nM sirolimus and 100 nM dasatinib for 48 hours. The PGLALA IgG has a silent Fc region and is a negative control. At 48 hours, blood was collected and lysed and the percentage of CD19⁺ B cell among live cells was measured by flow cytometry. Mean of n=2 donors.

FIG. 2. Effect of 100 nM ruxolitinib (ruxo), 100 nM sirolimus (siro), 100 nM dasatinib (dasa) on obinutuzumab-induced IFN-γ (FIG. 2A), IL-2 (FIG. 2B), TNF-α (FIG. 2C), IL-6 (FIG. 2D), IL-8 (FIG. 2E) and MCP-1 (FIG. 2F) for 100, 10, 1 and 0.1 μg/mL obinutuzumab in a whole blood assay. Fresh whole blood was incubated with 100, 10, 1 and 0.1 μg/mL obinutuzumab or PGLALA IgG in the presence and absence of 100 nM ruxolitinib, 100 nM sirolimus and 100 nM dasatinib for 48 hours. The PGLALA IgG has a silent Fc region and is a negative control. At 24 hours, serum was collected and cytokines were analyzed by Luminex. Mean of n=2 donors.

### EXAMPLES

The following are examples of methods and compositions of the invention. It is understood that various other aspects may be practiced, given the general description provided above.

### Example 1

### The JAK1/2 Inhibitor Ruxolitinib, the mTOR Inhibitor Sirolimus and the Src Inhibitor Dasatinib can Prevent FcγR Mediated Infusion Reaction

The CD20-targeting, effector-enhanced antibody obinutuzumab (Gazyva®) depletes B cells via FcγR signaling, and can be associated with a risk of infusion reaction characterized by cytokine release induced via FcγR signaling. To assess whether the mTOR inhibitor sirolimus, the JAK1/2 inhibitor ruxolitinib and the Src inhibitor dasatinib can prevent cytokine release induced via FcγR signaling, a whole blood assay using escalating doses of obinutuzumab in the presence and absence of sirolimus, ruxolitinib and dasatinib was conducted. A corresponding anti-CD20 IgG with a silent Fc portion (comprising “PGLALA” mutations L234A, L235A, P329G (Kabat EU numbering)) was used as a negative control (“PGLALA IgG”). In this assay, fresh whole blood is incubated together with concentrations of obinutuzumab ranging from 100 μg/mL to 0.1 μg/mL in the presence and absence of 100 nM sirolimus, 100 nM ruxolitinib and 100 nM dasatinib. To assess the impact of the different kinase inhibitors on cytokine release, serum was collected at 24 hours and cytokines were analyzed by Luminex. To assess the impact of the different kinase inhibitors on B cell depletion, blood was lysed at 48 hours and B cell depletion was measured by flow cytometry.

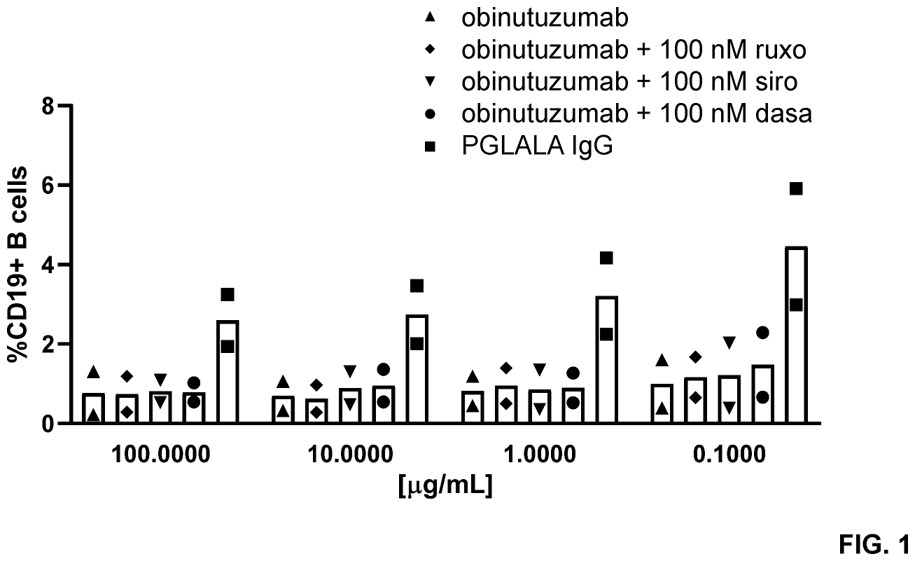

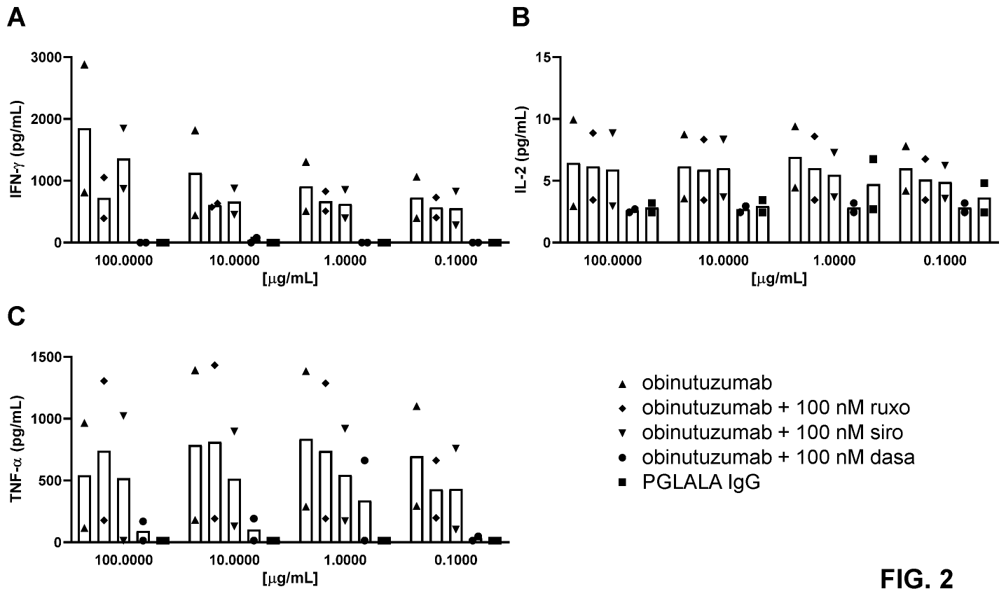

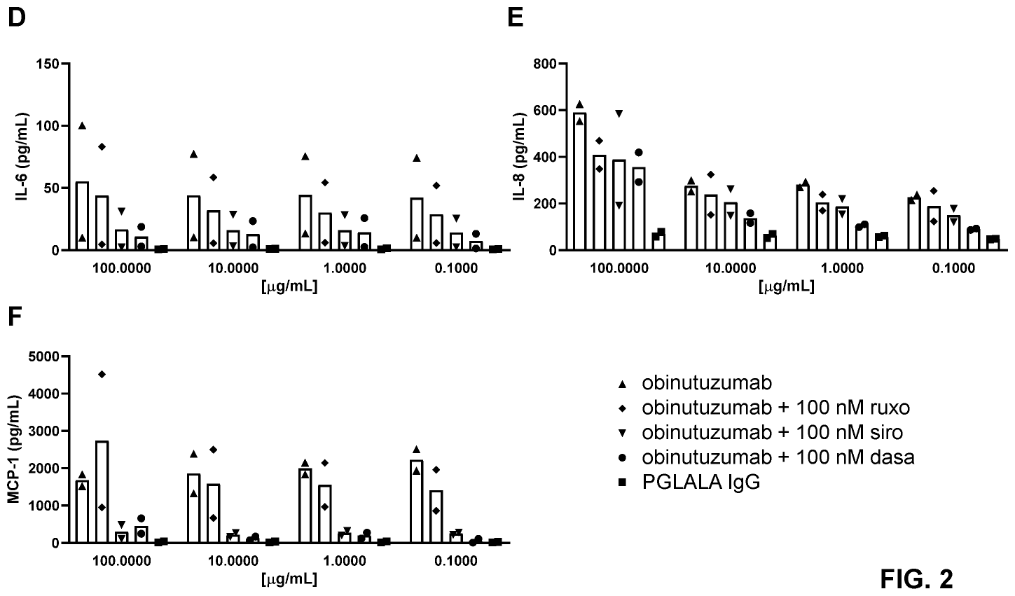

As a result, the treatment with 100 nM sirolimus, 100 nM ruxolitinib and 100 nM dasatinib did not prevent B cell depletion induced by obinutuzumab at 48 hours (FIG. 1). However, the treatment with 100 nM dasatinib strongly reduced the levels of IFN-γ, IL-2, TNF-α, IL-6, IL-6 and MCP-1 (FIGS. 2A-F). The treatment with 100 nM sirolimus and 100 nM ruxolitinib also reduced the levels of IFN-γ, TNF-α, IL-6, IL-6 and MCP-1 and to a lower extent IL-2 (FIGS. 2A-F). The mTOR inhibitors sirolimus, the JAK1/2 inhibitor ruxolitinib and the Src inhibitor dasatinib reduced cytokine release, with the Src inhibitor being the most potent among the three classes of kinase inhibitors in reducing cytokine release. Suprisingly, however, the kinase inhibitors (including dasatinib) did not interfere with obinutuzumab-induced B cell depletion. Hence, we propose that these kinase inhibitors could be potentially used to mitigate infusion reactions after the treatment with antibodies signaling via FcγR.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A natural killer (NK) cell engaging agent for use in the treatment of a disease in an individual, wherein said treatment comprises:
(a) administration of the NK cell engaging agent to the individual; and
(b) administration of an inhibitor of Src kinase (Src), Janus kinase (JAK) and/or mammalian target of rapamycin (mTOR) signaling to the individual.

2. A method for treatment of a disease in an individual, wherein said method comprises:
(a) administration of a natural killer (NK) cell engaging agent to the individual; and
(b) administration of an inhibitor of Src, JAK and/or mTOR signaling to the individual.

3. The method of claim 2, wherein the administration of the inhibitor of Src, JAK and/or mTOR signaling is for the prevention or mitigation of an adverse effect related to the administration of the NK cell engaging agent.

4. An inhibitor of Src, JAK and/or mTOR signaling for use in the prevention or mitigation of an adverse effect related to administration of an NK cell engaging agent to an individual.

5. A method for preventing or mitigating an adverse effect related to the administration of an NK cell engaging agent to an individual, wherein said method comprises administration of an inhibitor of Src, JAK and/or mTOR signaling to the individual.

6. The method of claim 2, wherein the inhibitor of Src, JAK and/or mTOR signaling is an Src inhibitor.

7. The method of claim 2, wherein the inhibitor of Src, JAK and/or mTOR signaling is an mTOR inhibitor.

8. The method of claim 2, wherein the inhibitor of Src, JAK and/or mTOR signaling is a JAK inhibitor.

9. The method of claim 2, wherein the administration of the inhibitor of Src, JAK and/or mTOR signaling causes inhibition of an adverse effect related to the administration of the NK cell engaging agent.

10. The method of claim 2, wherein the administration of the inhibitor of Src, JAK and/or mTOR signaling does not cause inhibition of a desired effect related to the administration of the NK cell engaging agent.

11. The method of claim 9, wherein the inhibition is a complete inhibition, or a clinically meaningful and/or statistically significant inhibition.

12. The method of claim 5, wherein the adverse effect is:
(i) cytokine release syndrome (CRS);
(ii) fever, hypotension and/or hypoxia; and/or
(iii) an elevated serum level of one of more cytokine.

13. The method of claim 5, wherein the administration of the inhibitor of Src, JAK and/or mTOR signaling is upon clinical manifestation of the adverse effect in the individual.

14. The method of claim 2, wherein the administration of the inhibitor of Src, JAK and/or mTOR signaling is:
(i) before, concurrent to, or after the administration of the NK cell engaging agent;
(ii) intermittently or continuously; and/or
(iii) oral or parenteral.

15. The method of claim 2, wherein the administration of the inhibitor of Src, JAK and/or mTOR signaling is associated with the first administration of the NK cell engaging agent.

16. The method of claim 2, wherein the administration of the NK cell engaging agent is:
(i) at an effective dose;
(ii) parenteral; and/or
(iii) the first administration of the NK cell engaging agent to the individual.

17. The method of claim 2, wherein the NK cell engaging agent is a CD16 binding agent.

18. The method of claim 2, wherein the NK cell engaging agent comprises an Fc region.

19. The method of claim 2, wherein the NK cell engaging agent is an antibody.

20. The method of claim 18, wherein the NK cell engaging agent binds to a target cell antigen.

21. The method of claim 20, wherein the target cell antigen is CD20.

22. The method of claim 2, wherein the NK cell engaging agent is an effector enhanced anti-CD20 antibody.

23. The method of claim 22, wherein the anti-CD20 antibody comprises a heavy chain 5 variable region comprising the heavy chain CDR (HCDR) 1 of SEQ ID NO: 2, the HCDR2 of SEQ ID NO: 3, and the HCDR3 of SEQ ID NO: 4; and a light chain variable region comprising the light chain CDR (LCDR) 1 of SEQ ID NO: 5, the LCDR2 of SEQ ID NO: 6 and the LCDR3 of SEQ ID NO: 7.

24. The method of claim 22, wherein anti-CD20 antibody comprises a heavy chain variable region sequence that is at least about 95%, 96%, 97%, 98%, 99% or 100% identical to the amino acid sequence of SEQ ID NO: 8 and/or a light chain variable region sequence that is at least about 95%, 96%, 97%, 98%, 99% or 100% identical to the amino acid sequence of SEQ ID NO: 9.

25. The method of claim 22, wherein the anti-CD20 antibody is engineered to have an increased proportion of non-fucosylated oligosaccharides in the Fc region as compared to a non-engineered antibody.

26. The method of claim 22, wherein the anti-CD20 antibody is obinutuzumab.

27. The method of claim 2, wherein the disease is cancer.
